(19) United States
(12) Patent Application Publication
(10) Pub. No.: US 2025/0000387 A1
(43) Pub. Date: Jan. 2, 2025

(54) FOOT INPUT SYSTEM, FOOT POSITION INDICATING INSTRUMENT, FOOT POSITION DETECTING DEVICE, AND IMAGE PROCESSING SYSTEM USING FOOT INPUT SYSTEM

(71) Applicant: Wacom Co., Ltd., Saitama (JP)

(72) Inventors: So KATO, Saitama (JP); Takeshi KOBORI, Saitama (JP)

(21) Appl. No.: 18/885,183

(22) Filed: Sep. 13, 2024

Related U.S. Application Data
(63) Continuation of application No. PCT/JP2023/009746, filed on Mar. 14, 2023.
(60) Provisional application No. 63/326,120, filed on Mar. 31, 2022.

(51) Int. Cl. A61B 5/103; A61B 5/00

(52) U.S. Cl. CPC A61B 5/1038; A61B 5/6807

(57) ABSTRACT

A foot input system includes a foot position indicating instrument configured to be located on a sole of a user, and a foot position detecting device. The foot position indicating instrument includes one or more position indication signal transmitting units configured to transmit a position indication signal. The foot position detecting device includes a position detecting unit that forms an operation surface on which the foot position indicating instrument moves and that receives a position indicated by the foot position indicating instrument; a position detecting sensor provided on a lower side of the position detecting unit to detect the position indicated by the foot position indicating instrument; and a detecting circuit configured to be supplied with a detection output from the position detecting sensor and detect and output the position indicated by the foot position indicating instrument. The position detecting unit has a concentric uneven structure.

### BACKGROUND

### Technical Field

The present disclosure relates to a system and device enabling information input using a foot portion of a user, and a system constructed to incorporate such system.

### Description of the Related Art

Recently, fields such as virtual reality (VR), augmented reality (AR), and mixed reality (MR) have developed rapidly due to improvements in the performance of computers and displays. In execution environments in these fields, instead of a conventional input device such as a mouse, a keyboard, or a gamepad, a special hand device that detects the movement of hands and fingers of a user may be used to operate a computer based on a sense of feeling similar to that the user feels in reality. It is thereby possible to reproduce a hand gesture, such as gripping and operating an object in hands, in a space created by the computer. Attempts have been made to bring the hand gesture of a user into digital content formed by the computer.

However, as the reproducibility of the operation by the hand gesture made by a user in reality is enhanced in the space created by the computer, it becomes more difficult to perform various operations by hand using an operation stick, an operation button, a touch panel, and the like. In addition, for example, a foot (legs) action such as a walking movement is substituted by a fingertip gesture or an arm movement gesture, which diminishes the sense of immersion critical in VR.

Accordingly, an input device that enables information input using a foot (leg) has been considered. For example, Japanese Patent Laid-open No. Hei 9-198188 describes an invention relating to a foot-operated input device that enables an input to be performed by a simple operation using only one foot. The foot-operated input device allows an indicated position to be changed by rotationally moving a ball provided to a sole side of the foot. It is provided with switches to be operated by toes of the foot to enable operations similar to left clicking and right clicking of a mouse.

In addition, Japanese Patent Laid-open No. 2016-174699 describes an invention relating to a game controller capable of not only measuring weight and a center of gravity but also detecting various movements such as stepping, walking, jumping, and squatting. The game controller detects a pressure distribution of a contact region contacted by a body part of a player (user) and detects a movement of the player on the basis of the shape of the distribution and a change in the shape when the player moves on a sheet having a plurality of pressure sensors.

### Prior Art

JP Publication H9-198188
JP Publication 2016-174699

### BRIEF SUMMARY

Recently, an environment in which high-quality VR can be used easily has been developed due to improvements in the performance and price reduction of VR apparatuses. For example, various improvements have been made in what is called a head-mounted display that is used in a state of being fitted to a head portion of the user to cover the eyes of the user. For example, there is a head-mounted display having high image quality and light weight, and a product including a whole body tracking technology in a head-mounted display. In addition, there is a head-mounted display that does not use an external information processing device (image processing device) such as a personal computer (PC), and instead includes a built-in image processing functionality and is further capable of wireless connection to an input device.

However, even now, there are various challenges facing their widespread use by many people. These mainly originate from a physiological phenomenon or are not addressable with existing devices, and thus cannot be solved by merely enhancing the performance of information processing devices such as a central processing unit (CPU) or a small-sized display. The present disclosure is directed to addressing those challenges.

For example, one challenge relates to a so-called “VR sickness” caused by a mismatch between information from a field of view and information based on a bodily sensation. When moving within a VR space by using an ordinary joystick held in a hand or the like, discomfort similar to motion sickness may occur because the movement of the field of view and the inertia of the bodily sensation do not match each other. That is, though moving within the VR space, the user is confined to merely operating the joystick and does not experience a traveling movement such as actual walking and, thus, a mismatch occurs between the movement of the field of view and the inertia of the bodily sensation. A novice not accustomed to a feeling of VR is considered particularly susceptible to VR sickness, which can be a hindrance to the introduction of the VR technology to more users.

Another challenge relates to how to use VR safely within a limited reduced space. When using VR, the field of view is blocked by the head-mounted display within a real space. The user may therefore be unable to understand where the user is located within a room in the real space. While using the VR technology in a standing state, the user's execution position may be gradually shifted, which may cause cable entanglement or a user running into a wall.

Another challenge relates to the complication caused by having to perform all operations of hands and feet movement in reality using only a VR hand controller held in the hands. With an ordinary VR controller, a large movement of the hands (arms) is used to indicate a moving operation of the body. Thus, as compared with a conventional computer game or the like, operations related to various movements become much more complicated.

Another challenge is that, during hand tracking, the VR hand controller cannot be held in the hands, and thus a movement operation becomes impossible. By using the hand tracking technology, it is possible to perform an operation within the VR space without holding the VR hand controller. However, because a movement operation using the joystick or the like cannot be performed, a movable range may be limited to the physical area of the room. In addition, even in a case of using the VR hand controller, a sense of immersion may be decreased because an operation is performed with the VR hand controller held in the hands. That is, because a movement operation that is originally performed by a foot is performed by a hand, natural hand movement cannot be performed, and the sense of immersion, which is an important characteristic of VR, may be diminished.

In addition, there are various problems with existing movement operation devices using a foot portion (leg portion). Specifically, there are already many kinds of operating input devices that receive an operating input by the foot portion of the user based on the user's actual walking or placing the foot on a device in a hemisphere shape and tilting the foot. However, the existing operating input devices for the foot portion often necessitate a large-sized device body, cause a sense of fatigue due to the repeated stepping movement required, and suffer from limited usable postures, an input delay, and a difficulty in performing a fine operation when only a slight movement is desired.

According to one aspect, the present disclosure is related to addressing the challenges described above and providing an environment in which many users can use the VR technology properly without a feeling of unease.

A foot input system includes a foot position indicating instrument configured to be located on a sole of a user, and a foot position detecting device including a position detecting unit that forms an operation surface on which the foot position indicating instrument moves and that receives a position indicated by the foot position indicating instrument. The foot position detecting device is configured to detect and output the indicated position on the position detecting unit. The foot position indicating instrument includes one or more position indication signal transmitting units configured to transmit a position indication signal. The foot position detecting device includes a position detecting sensor provided on a lower side of the position detecting unit to detect the position indicated by the foot position indicating instrument corresponding to the entire surface of the position detecting unit, and a detecting circuit configured to be supplied with a detection output from the position detecting sensor and detect the position indicated by the foot position indicating instrument on the position detecting unit. The position detecting unit has a concentric uneven structure.

According to the foot input system, the foot position indicating instrument includes the one or more position indication signal transmitting units that transmit a position indication signal. The foot position detecting device is provided with the position detecting sensor for detecting the position indicated by the foot position indicating instrument corresponding to the entire surface of the position detecting unit. The position detecting sensor is provided on the lower side of the position detecting unit forming the operation surface. The detecting circuit of the foot position detecting device is supplied with the detection output from the position detecting sensor, and detects the position indicated by the foot position indicating instrument on the position detecting unit. The position detecting unit of the foot position detecting device has a concentric uneven structure.

### BRIEF DESCRIPTION OF THE SEVERAL VIEWS OF THE DRAWINGS

FIG. 1 is a diagram of assistance in explaining an example of usage of a foot input system according to an embodiment;

FIG. 2 is a diagram of assistance in explaining a general configuration of an image processing system formed using the foot input system according to the embodiment;

FIGS. 3A, 3B, and 3C are diagrams of assistance in explaining an example of a configuration of a foot position indicating instrument;

FIG. 4 is a diagram of assistance in explaining a position indicated by the foot position indicating instrument;

FIGS. 5A, 5B, 5C, 5D, and 5E are diagrams of assistance in explaining manners of fitting the foot position indicating instrument to a foot portion of a user;

FIGS. 6A and 6B are diagrams of assistance in explaining an external configuration of a foot position detecting device;

FIG. 7 is a block diagram of assistance in explaining an internal configuration of the foot position detecting device;

FIGS. 8A, 8B, 8C, 8D, and 8E are diagrams of assistance in explaining the operation of the foot position indicating instrument on the foot position detecting device;

FIGS. 9A and 9B are diagrams of assistance in explaining positional relation between the foot position detecting device, a pivot foot of the user, and an indicating foot of the user fitted with the foot position indicating instrument;

FIGS. 10A, 10B, and 10C are diagrams of assistance in explaining an absolute coordinate system that can be used in the foot input system according to the embodiment;

FIGS. 11A, 11B, and 11C are diagrams of assistance in explaining output value complementation for an input value;

FIG. 12 is a diagram of assistance in explaining output value complementation for an input value;

FIGS. 13A, 13B, and 13C are diagrams of assistance in explaining output value complementation for an input value;

FIGS. 14A, 14B, and 14C are diagrams of assistance in explaining a case where the foot position indicating instrument is moved to the outside of a position detecting unit;

FIGS. 15A, 15B, and 15C are diagrams of assistance in explaining a relative coordinate system that can be used in the foot input system according to the embodiment;

FIGS. 16A, 16B, and 16C are diagrams of assistance in explaining control using only a relative coordinate value y (relative Y-axis control);

FIG. 17 is a diagram of assistance in explaining a concrete example of single axis control (relative Y-axis control) using the relative coordinate system;

FIG. 18 is a diagram of assistance in explaining a concrete example of X-Y-R-axis control using the absolute coordinate system;

FIG. 19 is a diagram of assistance in explaining the usage of stand-alone VR based on the X-Y-R-axis control using the absolute coordinate system;

FIG. 20 is a diagram illustrating an example of a state in which a foot position indicating instrument in another example is fitted to the foot portion of the user;

FIG. 21 is an external view of the foot position indicating instrument in another example;

FIGS. 22A, 22B, and 22C are diagrams of assistance in explaining an internal structure of the foot position indicating instrument in another example and the shape of an undersurface thereof;

FIGS. 23A, 23B, and 23C are diagrams of assistance in explaining an external constitution of a foot position detecting device in another example; and

FIGS. 24A, 24B, 24C, 24D, and 24E are diagrams of assistance in explaining the operation of the foot position indicating instrument on the foot position detecting device in another example.

### DETAILED DESCRIPTION

An embodiment of a system, a device, and a method according to the present disclosure will hereinafter be described with reference to the drawings.

### Usage Example of Foot Input System

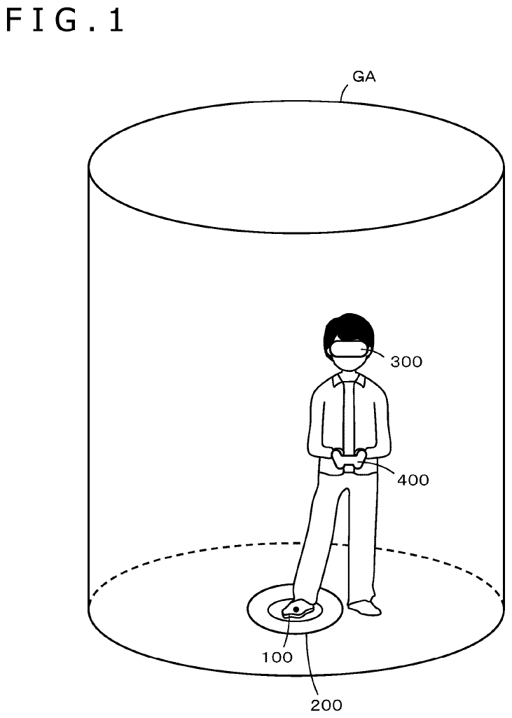

FIG. 1 is a diagram of assistance in explaining an example of usage of a foot input system according to an embodiment. The foot input system according to the embodiment includes a foot position indicating instrument 100 fitted to the sole of a foot portion of a user and a foot position detecting device 200 disposed on the lower side of the foot position indicating instrument 100. In the embodiment to be described in the following, an example of usage of the foot input system will be described by taking as an example a case where an image processing system is formed using the foot input system. As illustrated in FIG. 1, the foot position detecting device 200, a head-mounted display (HMD) 300, and a game controller 400 are connected to an image processing device 500 to be described later to form the image processing system.

The foot input system including the foot position indicating instrument 100 and the foot position detecting device 200 and the game controller 400 function as an input device that receives an indication input from the user, and supplies the received indication input to the image processing device 500. The HMD 300 is a display (display device) of a head mounted type. As illustrated in FIG. 1, the HMD 300 is fitted to a head portion of the user to cover both of the eyes of the user.

The image processing device 500 can form a three-dimensional space image (three-dimensional modeling image) over 360 degrees on the periphery of the user, as illustrated as a 360-degree image region “GA” in FIG. 1, to supply the three-dimensional space image to the HMD 300. In the present embodiment, the image processing device 500 functions as a so-called computer game machine that provides a game using the three-dimensional space image to the user.

### Example of Configuration of Image Processing System Using Foot Input System

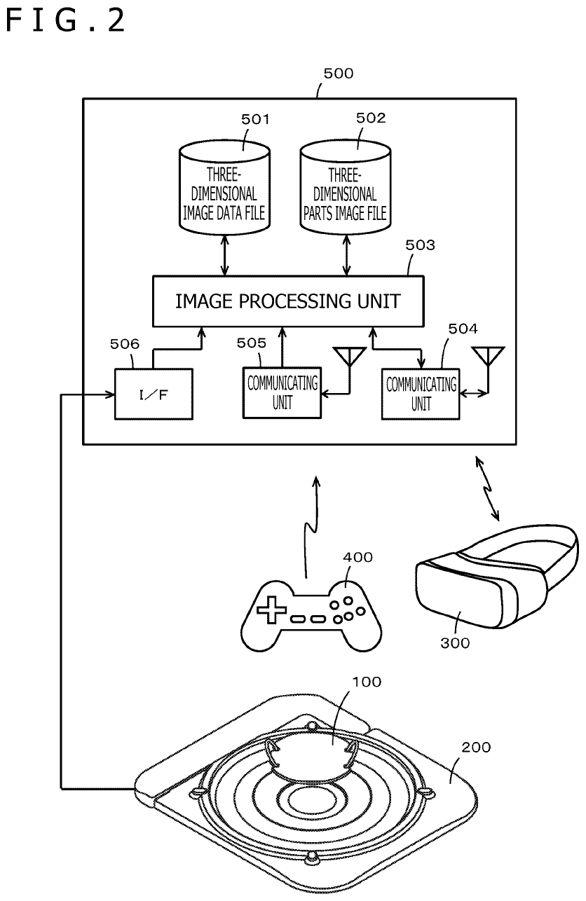

FIG. 2 is a diagram of assistance in explaining a general configuration of the image processing system configured using the foot input system according to the embodiment. As illustrated in FIG. 2, the image processing device 500 includes a three-dimensional image data file 501, a three-dimensional parts image file 502, an image processing unit 503, communicating units 504 and 505, and an interface (I/F) 506. The communicating unit 504 is to perform mutual wireless communication with the HMD 300. The communicating unit 505 is to receive an indication input from the game controller 400. The I/F 506 is to receive a detection output from the foot position detecting device 200 (i.e., an indication input using the foot position indicating instrument 100).

Thus, the image processing device 500 and the HMD 300 can bidirectionally communicate with each other wirelessly. In addition, the image processing device 500 and the game controller 400 are connected to each other wirelessly, and the image processing device 500 can receive at least an indication input from the game controller 400. In addition, the image processing device 500 and the foot position detecting device 200 are connected to each other via wire, and the image processing device 500 can receive a detection output from the foot position detecting device 200.

The HMD 300 and the game controller 400 can also be connected to the image processing device 500 via wire. However, the HMD 300 and the game controller 400 are worn or held by the user who may change the orientation of the body. Thus, the HMD 300 and the game controller 400 are desirably connected to the image processing device 500 wirelessly, which eliminates the risk of the use body getting entangled with a connection cord, for example. In addition, the foot position detecting device 200 and the image processing device 500 can also be connected to each other wirelessly. However, the foot position detecting device 200 does not move as the user moves, so no problem arises even when the foot position detecting device 200 is connected via wire.

The three-dimensional image data file 501 stores and retains three-dimensional image data forming the three-dimensional space image. The three-dimensional parts image file 502 stores and retains three-dimensional parts image data forming various three-dimensional parts images of, for example, an avatar and the like to be displayed within the three-dimensional space image. The image processing unit 503 forms three-dimensional space image data to be supplied to the HMD 300 by using the three-dimensional image data of the three-dimensional image data file 501 and the three-dimensional parts image data of the three-dimensional parts image file 502, and supplies the three-dimensional space image data to the HMD 300.

The HMD 300 includes a display HDP that displays the three-dimensional space image, and the HMD 300 includes, for example, a six-axis sensor formed of a three-axis gyro sensor and a three-axis angular velocity sensor, and can thereby detect a rotational direction and a rotational angle. Thus, the HMD 300 can display the three-dimensional space image corresponding to the three-dimensional image data from the image processing device 500 on the display HDP, and can transmit the detected rotational direction and the detected rotational angle to the image processing device 500. Therefore, when the user wearing the HMD 300 on the head portion performs a movement of rotating the head portion to face left or right, looking up, or looking down, the six-axis sensor mounted in the HMD 300 detects a degree and a direction of the rotation, and notifies the degree and the direction of the rotation to the image processing device 500.

The image processing unit 503 of the image processing device 500 identifies a direction in which both eyes of the user are directed on the basis of the detection output from the six-axis sensor of the HMD 300, forms three-dimensional space image data in the viewing direction of the user, and supplies the three-dimensional space image data to the HMD 300. The user can thereby view a three-dimensional space image corresponding to the direction in which both eyes of the user are directed through the display HDP of the HMD 300.

In addition, the image processing unit 503 of the image processing device 500 can generate three-dimensional space image data to which a change is made, such as when the avatar is throwing a ball or firing a gun within the three-dimensional space image displayed on the display of the HMD 300, according to an instruction input from the game controller 400, and supply the three-dimensional space image data to the HMD 300. Thus, the three-dimensional space image that changes according to the instruction input that is input via the game controller 400 can be viewed through the display HDP of the HMD 300.

Further, the image processing unit 503 of the image processing device 500 enables movement of the avatar or a viewpoint in a three-dimensional space (VR space) displayed on the display of the HMD 300 according to the detection output from the foot position detecting device 200. Specifically, when the foot position indicating instrument 100 is slidingly moved in a direction in which the front surface of the body is facing in the longitudinal direction of a foot portion on the foot position detecting device 200, the image processing device 500 can be instructed to advance the avatar or the viewpoint in the three-dimensional space. Conversely, when the foot position indicating instrument 100 is slidingly moved in a direction in which the back surface of the body is facing in the longitudinal direction of the foot portion on the foot position detecting device 200, the image processing device 500 can be instructed to back the avatar or the viewpoint in the three-dimensional space.

In addition, suppose that the foot position indicating instrument 100 is slidingly moved in a leftward direction of the body in a direction intersecting the longitudinal direction of the foot portion on the foot position detecting device 200. In this case, the image processing device 500 can be instructed to move the avatar or the viewpoint to a left side in the three-dimensional space. Conversely, suppose that the foot position indicating instrument 100 is slidingly moved in a rightward direction of the body in the direction intersecting the longitudinal direction of the foot portion on the foot position detecting device 200. In this case, the image processing device 500 can be instructed to move the avatar or the viewpoint to a right side in the three-dimensional space.

Thus, as illustrated in FIG. 1, the user wearing the HMD 300 on the head portion, holding the game controller 400 in hands, and placing a right foot fitted with the foot position indicating instrument 100 on the foot position detecting device 200 can enjoy the game using the three-dimensional space image. In this case, the user can change the viewing direction by performing a movement of rotating the head portion left or right, looking up, or looking down, and thereby correspondingly change the three-dimensional space image displayed on the display of the HMD 300.

In addition, the display of a three-dimensional image object such as the avatar can be changed within the displayed three-dimensional space image by operating the game controller 400. Further, a movement of the avatar or the viewpoint can be performed in the three-dimensional space (in the VR space) by moving the foot position indicating instrument on the foot position detecting device 200.

The rotation of the head portion is not limited to a case where only the head portion is rotated but also includes a case where the entire body of the user is rotated. Hence, as illustrated in FIG. 1, the user can enjoy the game by using the three-dimensional space image on the entire periphery of the 360-degree image region GA while freely performing a rotational movement such as, for example, changing the orientation of the user's body by rotating the entire body. Furthermore, for the three-dimensional space image in any direction, a movement of the avatar or the viewpoint position within the three-dimensional space can be performed through the foot position indicating instrument 100 and the foot position detecting device 200. Thus, it is possible to enjoy the game while dynamically changing the three-dimensional space image based on the rotation of the head portion and the operation of the foot portion.

### Example of Configuration of Foot Position Indicating Instrument 100

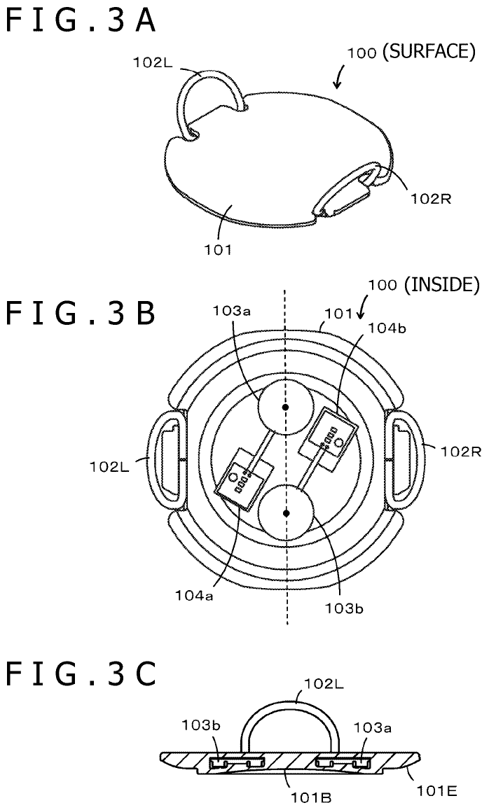

FIGS. 3A to 3C are diagrams of assistance in explaining an example of a configuration of the foot position indicating instrument 100. Of FIGS. 3A to 3C, FIG. 3A is an external view of the foot position indicating instrument 100, FIG. 3B is a view illustrating an internal configuration of the foot position indicating instrument 100, and FIG. 3C is a sectional view of the foot position indicating instrument 100. As illustrated in FIG. 3A, the foot position indicating instrument 100 includes a main body unit 101 and belt holding units 102L and 102R. The main body unit 101 is a substantially circular plate-shaped body having a diameter of approximately 7 to 8 cm, for example, and a predetermined thickness. As illustrated in FIG. 3A, each of the belt holding units 102L and 102R is a ring-shaped unit attached so as not to come off to the left or right of the main body unit 101 easily.

The interior of the main body unit 101 of the foot position indicating instrument 100 is exposed when an upper surface plate (top plate) is removed. As illustrated in FIG. 3B, coils 103a and 103b and circuit boards 104a and 104b are mounted within the main body unit 101. The coils 103a and 103b are obtained by forming coils having N turns (N is an integer of 1 or more) into a circular and flat shape (flat and thin shape). Hence, the coils 103a and 103b generate a magnetic field in a direction intersecting the bottom surface and the upper surface of the main body unit 101. The circuit boards 104a and 104b are formed with circuit parts such as capacitors mounted thereon. In the present embodiment, as illustrated in FIG. 3B, the coil 103a and the circuit board 104a constitute one resonance circuit, and the coil 103b and the circuit board 104b constitute another resonance circuit.

In the present embodiment, the resonance circuit constituted by the coil 103a and the circuit board 104a and the resonance circuit constituted by the coil 103b and the circuit board 104b have resonance frequencies different from each other. Each of the two resonance circuits can be thereby distinguished from the other. The main body unit 101 also includes a first recessed portion fitted with the resonance circuit constituted by the coil 103a and the circuit board 104a and a second recessed portion fitted with the resonance circuit constituted by the coil 103b and the circuit board 104b. The positions of the coils 103a and 103b and the circuit boards 104a and 104b within the main body unit 101 are fixed by fitting the resonance circuits to the corresponding first and second recessed portions, respectively. FIG. 3B illustrates a state in which the resonance circuits are fitted in the first and second recessed portions.

Thus, as illustrated in FIG. 3B, the two resonance circuits can be mounted within the main body unit 101 such that the center of the coil 103a and the center of the coil 103b are located on a straight line. Hence, when sectioning is performed at a position indicated by a dotted line in FIG. 3B, and the belt holding unit 102R side is removed, a configuration as illustrated in FIG. 3 is revealed in which the coil 103a and the coil 103b are mounted at a defined interval from each other within the main body unit 101. The coils 103a and 103b and the circuit boards 104a and 104b are protected appropriately without being exposed from the bottom surface or the upper surface of the main body unit 101.

As illustrated in FIG. 3C, the bottom surface 101B of the main body unit 101 forms a spherical recessed portion by being recessed in a spherical shape toward the upper surface. The bottom surface 101B forming the spherical recessed portion is a part to be fitted with a central part (a central protruding portion) of the position detecting unit of the foot position detecting device 200 to be described later. Further, an outer circumferential portion bottom surface 101E of the main body unit 101 has a smooth arc shape. As described later, the outer circumferential portion bottom surface 101E has a functional purpose when it catches (is engaged with) an outer edge portion of the position detecting unit of the foot position detecting device 200. The main body unit 101 as a whole is formed by using a raw material superior in the wear resistance property and the sliding property such as, for example, a polyacetal (POM) resin.

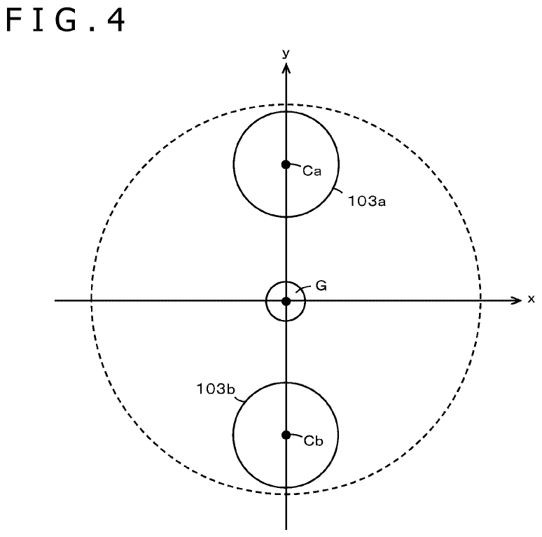

FIG. 4 is a diagram of assistance in explaining a position indicated by the foot position indicating instrument 100. As illustrated in FIG. 4, a straight line connecting together the centers of the respective coils 103a and 103b of the resonance circuits mounted in the foot position indicating instrument 100 is a y-axis of the foot position indicating instrument 100. As illustrated in FIG. 4, a middle position between a center Ca of the coil 103a and a center Cb of the coil 103b on the y-axis is a midpoint G. A straight line passing through the midpoint G and orthogonal to the y-axis is an x-axis of the foot position indicating instrument 100. In the present embodiment, the midpoint G of the foot position indicating instrument 100 is an indicated position (detected position) on a position detecting sensor of the foot position detecting device 200. In addition, the foot position detecting device 200 calculates a rotational angle of the foot position indicating instrument 100 with respect to coordinates on the position detecting sensor.

### Another Example of Configuration of Foot Position Indicating Instrument 100

In the case of the foot position indicating instrument 100 described with reference to FIGS. 3A to 3C and FIG. 4, description has been made assuming that the foot position indicating instrument 100 is configured to be mounted with two resonance circuits, that is, the resonance circuit constituted by the coil 103a and the circuit board 104a and the resonance circuit constituted by the coil 103b and the circuit board 104b. However, the disclosure is not so limited. A configuration simply including one resonance circuit constituted by one coil and one circuit board is also possible, as will be apparent to those skilled in the art.

In addition, a foot position indicating instrument mounted with a plurality (more than 2) of resonance circuits having different resonance frequencies can be configured. For example, a configuration may be adopted in which three resonance circuits are mounted and the centers of coils of the respective resonance circuits are located at the vertices of a regular triangle. In this case, for example, a straight line obtained by including a base and extending the base becomes the x-axis of the foot position indicating instrument, and a straight line that passes through a vertex other than both ends of the base and is orthogonal to the base becomes the y-axis of the foot position indicating instrument. The center of the regular triangle, for example, can be set as an indicated position.

In addition, a configuration may be adopted in which four resonance circuits are mounted and the centers of coils of the respective resonance circuits are located at four vertices of a square. In this case, for example, one diagonal line is the x-axis of the foot position indicating instrument, and another diagonal line is the y-axis of the foot position indicating instrument. In this example, the center of the square can be set as an indicated position. Thus, the foot position indicating instrument can be configured to be mounted with a plurality of resonance circuits. Further, more resonance circuits can be mounted when the mounting is possible.

### Manner of Fitting Foot Position Indicating Instrument 100

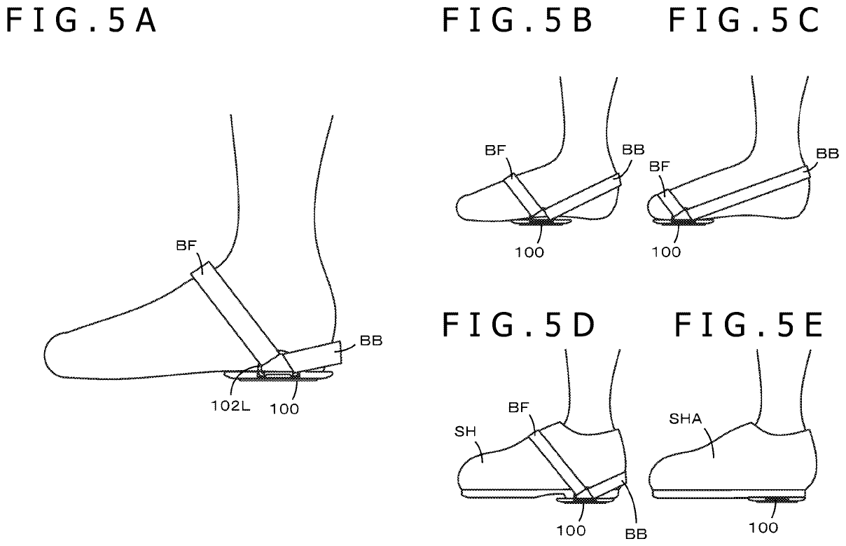

FIGS. 5A to 5E are diagrams of assistance in explaining manners of fitting the foot position indicating instrument 100 to the foot portion of the user. As illustrated in FIG. 5A, the foot position indicating instrument 100 having an external appearance illustrated in FIG. 3A is fixed to the foot portion (sole) of the user with a front side belt BF and a rear side belt BB passed through the belt holding unit 102L and the belt holding unit 102R, which is not shown in FIG. 5A. As illustrated in FIG. 5A, the front side belt BF is a belt fastened on a front side of the foot portion of the user (instep of the foot), and the rear side belt BB is a belt fastened on a rear side of the foot portion of the user (side surfaces in the rear of a heel).

As illustrated in FIG. 5A, the foot position indicating instrument 100 can be fitted to a heel portion, which is a rear side part of the sole of the user, by the front side belt BF and the rear side belt BB. By adjusting the lengths of the front side belt BF and the rear side belt BB, it is possible to fit the foot position indicating instrument 100 to a plantar arch portion, which is a central part of the sole of the user, as illustrated in FIG. 5B, or fit the foot position indicating instrument 100 to a toe side portion, which is a front side part of the foot portion of the user. Needless to say, as illustrated in FIGS. 5A, 5B, and 5C, the foot position indicating instrument 100 can be fitted directly to the foot portion of the user or fitted indirectly to the foot portion wearing a sock, for example. The fitting position of the foot position indicating instrument 100 can be adjusted according to the user. In addition, as illustrated in FIG. 5D, the foot position indicating instrument 100 can also be fitted to the foot portion of the user in a state of wearing footwear SH such as a sports shoe in the manners illustrated in FIGS. 5A, 5B, and 5C.

In addition, as illustrated in FIG. 5E, the foot position indicating instrument 100 may be fixed to the sole of footwear SHA such as a shoe or a slipper. The foot position indicating instrument 100 may be incorporated in the footwear itself. The foot position indicating instrument 100 can be thereby fitted to the foot portion of the user when the user puts on the footwear SHA to which the foot position indicating instrument 100 is fixed. In addition, also in the case where the foot position indicating instrument 100 is fixed to the footwear, the foot position indicating instrument 100 can be not only fitted to the heel portion as illustrated in FIG. 5E but also fixed to the plantar arch portion as in the case illustrated in FIG. 5B or fixed to the toe side portion as in the case illustrated in FIG. 5C.

### Example of Configuration of Foot Position Detecting Device 200

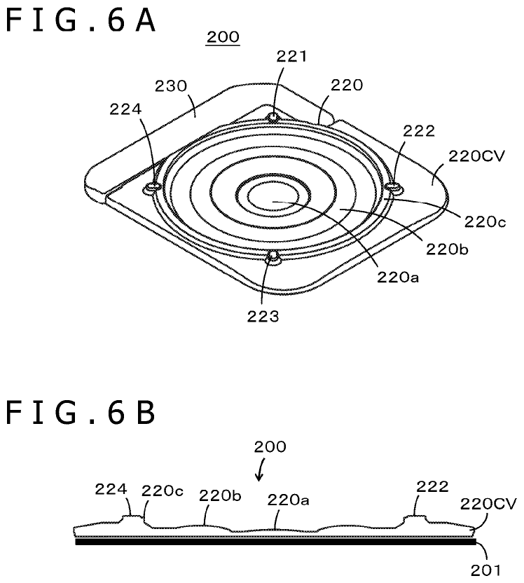

Description will next be made of the foot position detecting device 200 that detects an indicated position indicated by the foot position indicating instrument 100 mounted with the resonance circuits as described above and the rotational angle of the foot position indicating instrument 100. FIGS. 6A and 6B are diagrams of assistance in explaining an external configuration of the foot position detecting device 200. Of FIGS. 6A and 6B, FIG. 6A is a perspective view of the foot position detecting device 200, and FIG. 6B is a sectional view of the foot position detecting device 200.

As illustrated in FIG. 6A, the external appearance of the foot position detecting device 200 includes a quadrangular position detecting unit cover 220CV having a large circular position detecting unit 220 formed therein and a circuit mounted unit 230 formed in an L-shape in an upper left part. As indicated by a plurality of concentric circles, the position detecting unit 220 has an overall shape of a dish that is depressed (recessed) stepwise from the outside to the inside. That is, the inside of the position detecting unit 220 has a concentric uneven (not flat) structure. In the present embodiment, the position detecting unit 220 has a three-stage structure of a highest outer portion, a lowest inner portion, and an intermediate portion located therebetween.

More specifically, a lowest circular part located in the center of the position detecting unit 220 is a central convex portion 220a that slightly bulges upward in a spherical shape. On the periphery of the central convex portion 220a is a doughnut-shaped convex portion 220b at a position somewhat higher than the central convex portion 220a, the doughnut-shaped convex portion 220b having a predetermined width and slightly bulging upward on the central convex portion 220a side. On the periphery of the doughnut-shaped convex portion 220b is an outer wall-shaped protruding portion 220c that is slightly higher than the doughnut-shaped convex portion 220b and thereby forms an outer wall along an outer edge. That is, the central convex portion 220a corresponds to the inner portion, the doughnut-shaped convex portion 220b corresponds to the intermediate portion, and the outer wall-shaped protruding portion 220c corresponds to the outer portion.

In addition, as illustrated in FIG. 6A, direction detection protruding portions 221, 222, 223, and 224 are provided in four directions so as to be laid on the outer wall-shaped protruding portion 220c. In the foot position detecting device 200, a straight line connecting the direction detection protruding portion 221 and the direction detection protruding portion 223 to each other is a Y-axis, and a straight line connecting the direction detection protruding portion 222 and the direction detection protruding portion 224 to each other is an X-axis. Hence, the Y-axis and the X-axis are orthogonal to each other at the center of the position detecting unit 220.

FIG. 6B is a sectional view of the foot position detecting device 200 viewed when the foot position detecting device 200 in a state illustrated in FIG. 6A is sectioned by the X-axis, which is a straight line connecting the direction detection protruding portion 222 and the direction detection protruding portion 224 to each other, and a front side part is removed. As illustrated in FIG. 6B, a position detecting sensor 201 is provided on the lower side of the position detecting unit cover 220CV. Incidentally, in FIG. 6B, in order to illustrate the configuration clearly, the position detecting sensor 201 part is illustrated in a filled-in state, and the position detecting unit cover 220CV part is illustrated as an outline.

Referring to FIG. 6B, description will be made of a configuration of the position detecting unit 220 of the position detecting unit cover 220CV. As has been described above, the position detecting unit 220 as a whole has a shape of a dish that is recessed stepwise from the outside (outer wall-shaped protruding portion 220c) to the inside. A lowest circular part of the position detecting unit 220 is recognized as the central convex portion 220a that bulges upward in a spherical shape. The outside periphery of the central convex portion 220a is recognized as the doughnut-shaped convex portion 220b, which is somewhat higher than the central convex portion 220a, has a predetermined width, and bulges upward on the central convex portion 220a side.

The outside periphery of the doughnut-shaped convex portion 220b is recognized as the outer wall-shaped protruding portion 220c, which is slightly higher than the doughnut-shaped convex portion 220b and thereby forms an outer wall along the outer edge of the doughnut-shaped convex portion 220b. Thus, the position detecting unit 220 formed in the position detecting unit cover 220CV according to the present embodiment has a circular shape, is recessed stepwise from the outside to the inside, and has a three-stage structure of the central convex portion 220a, the doughnut-shaped convex portion 220b, and the outer wall-shaped protruding portion 220c.

Incidentally, the position detecting unit cover 220CV including the position detecting unit 220 is detachable from basic constituent parts (basic casing parts) of the foot position detecting device 200 including the position detecting sensor 201 and the circuit mounted unit 230 mounted with a position detecting circuit 202. That is, the position detecting unit cover 220CV including the position detecting unit 220 is configured as an attachment accessory part in the foot position detecting device 200. Thus, if the position detecting unit 220 is degraded by being rubbed through contact with the foot position indicating instrument 100, the position detecting unit 220 can be replaced easily. It is thereby possible to maintain a high performance of the foot position detecting device 200 by merely replacing the position detecting unit cover 220CV without changing the position detecting sensor 201 or the circuit mounted unit 230. Here, while description has been made assuming that the entire position detecting unit cover 220CV is detachable, it is sufficient that at least the position detecting unit 220 is made detachable.

### Internal Configuration of Foot Position Detecting Device 200

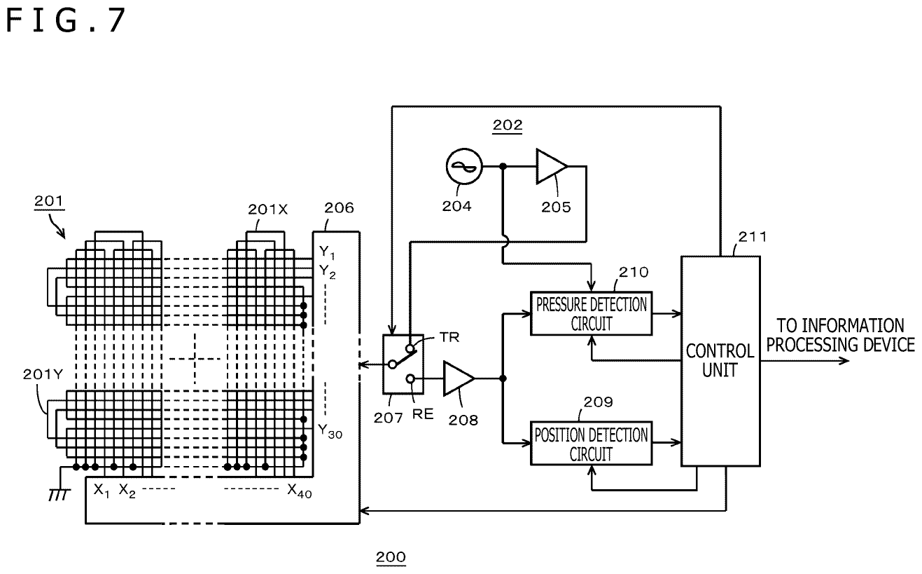

Description will next be made of an internal configuration of the foot position detecting device 200 including the position detecting sensor 201 located on the lower side of the position detecting unit cover 220CV as illustrated in FIG. 6B. FIG. 7 is a block diagram of assistance in explaining an internal configuration of the foot position detecting device 200. The foot position detecting device 200 includes an electromagnetic induction system applied thereto to detect a position indicated by and the rotational angle of the foot position indicating instrument 100 including the resonance circuits, as described above.

As illustrated in FIG. 7, when broadly divided, the foot position detecting device 200 includes the position detecting sensor 201 and the position detecting circuit 202. The position detecting sensor 201 is formed by layering an X-axis direction loop coil group 201X and a Y-axis direction loop coil group 201Y. As also illustrated in FIG. 7, the position detecting sensor 201 is used in a state of being disposed at a foot of the user and located on the lower side of the foot position indicating instrument 100.

Each of loop coils X1 to X40 of the X-axis loop coil group 201X and loop coils Y1 to Y30 of the Y-axis loop coil group 201Y constituting electrodes of the position detecting sensor 201 may have one turn, or may have two turns or more, that is, a plurality of turns. In addition, the number of loop coils of each of the loop coil groups 201X and 201Y can be suitably set according to the size of the position detecting sensor 201.

The position detecting circuit 202 includes an oscillator 204, a current driver 205, a selecting circuit 206, a switching connecting circuit 207, a receiving amplifier 208, a position detection circuit 209, a pressure detection circuit 210, and a control unit 211. The control unit 211 is a microprocessor formed by connecting a central processing unit (CPU), a read only memory (ROM), a random access memory (RAM), a nonvolatile memory, and the like. The control unit 211 controls selection of a loop coil in the selecting circuit 206 and switching of the switching connecting circuit 207, and controls processing timing in the position detection circuit 209 and the pressure detection circuit 210.

The X-axis direction loop coil group 201X and the Y-axis direction loop coil group 201Y of the position detecting sensor 201 are connected to the selecting circuit 206. The selecting circuit 206 sequentially selects one loop coil of the two loop coil groups 201X and 201Y. The oscillator 204 generates an alternating-current signal of a frequency f0. The oscillator 204 supplies the generated alternating-current signal to the current driver 205 and the pressure detection circuit 210. The current driver 205 converts the alternating-current signal supplied from the oscillator 204 into a current, and sends out the current to the switching connecting circuit 207.

The switching connecting circuit 207 switches between connection destinations (a transmitting side terminal TR and a receiving side terminal RE) to which the loop coil selected by the selecting circuit 206 is connected under control of the control unit 211. Of the connection destinations, the transmitting side terminal TR is connected with the current driver 205, and the receiving side terminal RE is connected with the receiving amplifier 208. Then, when a signal is to be transmitted from the position detecting sensor 201, the switching connecting circuit 207 is switched to the terminal TR side. Conversely, when the position detecting sensor 201 is to receive an external signal, the switching connecting circuit 207 is switched to the terminal RE side.

Then, when the switching connecting circuit 207 is switched to the terminal TR side, the current from the current driver 205 is supplied to the loop coil selected by the selecting circuit 206. Consequently, a magnetic field is generated by the loop coil to interact with the resonance circuits included in the foot position indicating instrument 100 facing the loop coil, so that a signal (radio wave) can be transmitted.

When the switching connecting circuit 207 is switched to the terminal RE side, on the other hand, an induced voltage occurring in the loop coil selected by the selecting circuit 206 is sent to the receiving amplifier 208 via the selecting circuit 206 and the switching connecting circuit 207. The receiving amplifier 208 amplifies the induced voltage supplied from the loop coil, and sends out the induced voltage to the position detection circuit 209 and the pressure detection circuit 210.

Specifically, radio waves (position indication signals) transmitted from position indication units 101U and 103U of the foot position indicating instrument 100 cause an induced voltage in each loop coil of the X-axis direction loop coil group 201X and the Y-axis direction loop coil group 201Y. The position detection circuit 209 detects the induced voltage occurring in the loop coil (i.e., a received signal), converts the detected output signal into a digital signal, and outputs the digital signal to the control unit 211. The control unit 211 calculates the coordinate values of positions indicated in the X-axis direction and the Y-axis direction by the position indication signals from the position indication units 101U and 103U on the basis of the digital signal from the position detection circuit 209, that is, the level of the voltage value of the induced voltage occurring in each loop coil.

In a case where a pressure sensor is mounted in the foot position indicating instrument 100, the detection output of the pressure sensor can be superimposed on the signal sent out from the foot position indicating instrument 100. Therefore, the pressure detection circuit 210 synchronously detects the output signal of the receiving amplifier 208 using the alternating-current signal from the oscillator 204, obtains a signal having a level corresponding to a phase difference (frequency shift) between these signals, converts the signal corresponding to the phase difference (frequency shift) into a digital signal, and outputs the digital signal to the control unit 211. The control unit 211 can detect a pressure applied to the pressure sensor of the foot position indicating instrument 100 on the basis of the digital signal from the pressure detection circuit 210, that is, the signal level corresponding to the phase difference (frequency shift) between the transmitted radio wave and the received radio wave.

### Operation of Foot Position Indicating Instrument 100 on Foot Position Detecting Device 200

As illustrated in FIG. 1 and FIG. 2, the foot position indicating instrument 100 is fitted to a foot portion of the user, and is used on an operation surface (position detecting unit 220) of the foot position detecting device 200. As described with reference to FIG. 3C, the bottom surface 101B of the main body unit 101 of the foot position indicating instrument 100 is recessed in a spherical shape toward the upper surface, to thereby form a spherical recessed portion. On the other hand, as described with reference to FIGS. 6A and 6B, the position detecting unit 220 of the foot position detecting device 200, which serves as the operation surface on which the foot position indicating instrument 100 is operated, has a three-stage structure of the central convex portion 220a, the doughnut-shaped convex portion 220b, and the outer wall-shaped protruding portion 220c. The foot position indicating instrument 100 and the foot position detecting device 200 thusly configured provide a superior foot-based input system that has not existed before. In the following, the operation of the foot position indicating instrument 100 on the foot position detecting device 200 will be described.

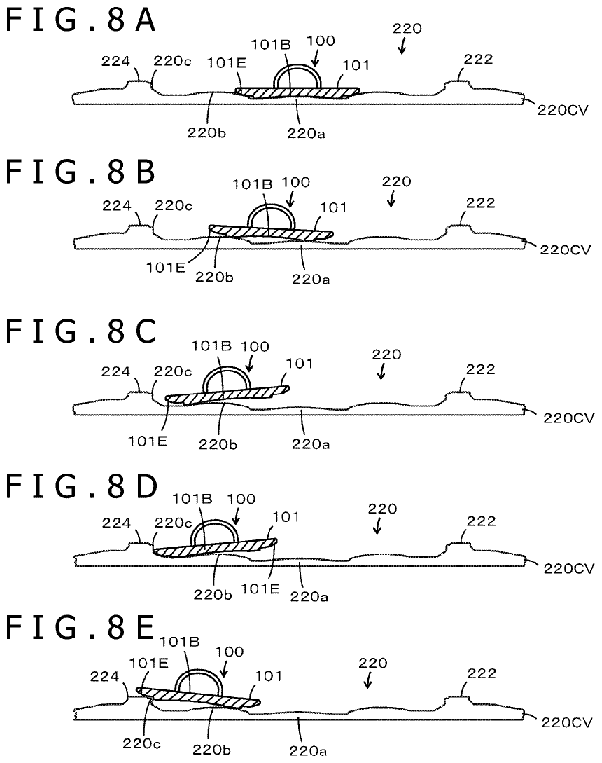

FIGS. 8A to 8E are diagrams of assistance in explaining the operation of the foot position indicating instrument 100 on the foot position detecting device 200, and illustrate a section of the position detecting unit cover 220CV of the foot position detecting device 200 and a section of the main body unit 101 of the foot position indicating instrument 100. Incidentally, in order to clarify a distinction therebetween, the section of the position detecting unit cover 220CV of the foot position detecting device 200 is illustrated in an outlined state, and the section of the main body unit 101 of the foot position indicating instrument 100 is illustrated in a hatched state.

An internal surface shape of the spherical recessed portion of the bottom surface 101B of the main body unit 101 and an external surface shape of the central convex portion 220a of the position detecting unit 220 are made to coincide with each other. Therefore, as illustrated in

FIG. 8A, when the foot position indicating instrument 100 is located on the central convex portion 220a of the position detecting unit 220 on the inside of the outer wall-shaped protruding portion 220c of the foot position detecting device 200, the central convex portion 220a of the position detecting unit 220 fits into and catches the bottom surface 101B forming the spherical recessed portion of the main body unit 101. The user can thereby clearly grasp the position of the user in a real space. Furthermore, a horizontal slipping of the foot position indicating instrument 100 on the position detecting unit 220 is prevented, and the foot position indicating instrument 100 can be rotated smoothly over the entire circumference of 360 degrees. That is, the user fitted with the foot position indicating instrument 100 can smoothly and stably rotate about the foot fitted with the foot position indicating instrument 100.

In addition, in the position detecting unit 220 of the foot position detecting device 200, the doughnut-shaped convex portion 220b that has a predetermined width and slightly bulges upward on the central convex portion 220a side is present on the periphery of the central convex portion 220a and at a position somewhat higher than the central convex portion 220a. Therefore, as illustrated in FIG. 8B, when the foot position indicating instrument 100 is moved outward from the central convex portion 220a of the position detecting unit 220, the doughnut-shaped convex portion 220b blocks a sudden movement of the foot position indicating instrument 100, and allows the foot position indicating instrument 100 to be moved slowly.

Conversely, when the foot position indicating instrument 100 is moved from the doughnut-shaped convex portion 220b side to the central convex portion 220a side, the foot position indicating instrument 100 can be moved so as to slide down to the central convex portion 220a. This structure facilitates a quick return of the foot position indicating instrument 100 located on the doughnut-shaped convex portion 220b to the central convex portion 220a at the center. The structure therefore allows the foot position indicating instrument 100 to return to the central convex portion 220a quickly in any direction over 360 degrees about the central convex portion 220a when the foot position indicating instrument 100 is moved from the doughnut-shaped convex portion 220b side to the central convex portion 220a side. Thus, the doughnut-shaped convex portion 220b located in an intermediate portion of the position detecting unit 220 allows a slow movement from the central convex portion 220a to the outside and a quick return from the doughnut-shaped convex portion 220b side to the central convex portion 220a.

As described above with reference to FIG. 8B, when the foot position indicating instrument 100 is moved outward from the central convex portion 220a of the position detecting unit 220, the doughnut-shaped convex portion 220b suppresses a sudden movement of the foot position indicating instrument 100, and allows the foot position indicating instrument 100 to be moved slowly. However, the foot position indicating instrument 100 is moved by applying a slight force to the foot portion fitted with the foot position indicating instrument 100. Therefore, as illustrated in FIG. 8C, when the foot position indicating instrument 100 goes over the doughnut-shaped convex portion 220b, the force may be applied so as to move the foot position indicating instrument 100 further outward.

In this case, as illustrated in FIG. 8D, the side surface of the main body unit 101 of the foot position indicating instrument 100 abuts against the inner side surface of the outer wall-shaped protruding portion 220c of the position detecting unit 220, so that the foot position indicating instrument 100 can be prevented from moving further outward. In addition, as illustrated in FIG. 8E, suppose that the outer circumferential portion bottom surface 101E of the foot position indicating instrument 100 has moved onto the outer wall-shaped protruding portion 220c of the position detecting unit 220. Even in this case, because the outer circumferential portion bottom surface 101E of the foot position indicating instrument 100 has a smooth arc shape, the foot position indicating instrument 100 that has moved onto the outer wall-shaped protruding portion 220c of the position detecting unit 220 can slide down, and the smooth arc shape facilitates a return of the foot position indicating instrument 100 to the inside of the position detecting unit 220.

In addition, as also described above, the position detecting unit 220 of the foot position detecting device 200 has a circular shape, is recessed stepwise from the outside to the inside, and has a three-stage structure of the central convex portion 220a, the doughnut-shaped convex portion 220b, and the outer wall-shaped protruding portion 220c. The presence of the doughnut-shaped convex portion 220b in the intermediate portion of the position detecting unit 220, in particular, can effectively prevent the foot position indicating instrument 100 from springing out to the outside by momentum as compared with a case where the entire position detecting unit 220 has a flat shape (flat plate) or a case where the entire position detecting unit 220 has a simple conical shape.

In addition, as illustrated in FIG. 8E, the outer wall-shaped protruding portion 220c of the position detecting unit 220 and the doughnut-shaped convex portion 220b in the intermediate portion thereof form a structure that facilitates a smooth movement of the foot position indicating instrument 100 over the entire circumference of 360 degrees along the outer circumference of the position detecting unit 220. Thus, an input operation can be performed stably at all times without the foot position indicating instrument 100 getting dislodged from the inside of the position detecting unit 220. Hence, it is possible to avoid the inconvenience of the user continuously shifting his/her position in the real space without realizing it to eventually run into a wall, for example.

Further, as illustrated in FIG. 6A, the position detecting unit 220 of the foot position detecting device 200 has the direction detection protruding portions 221, 222, 223, and 224 arranged at defined positions on the outer wall-shaped protruding portion 220c. In FIG. 6A, a total of four direction detection protruding portions 221, 222, 223, and 224 are provided at intervals of 90 degrees. Thus, the user can sense the swelling of the direction detection protruding portions 221, 222, 223, and 224 through his/her sole, to thereby recognize each reference direction through the sole. Incidentally, the height of the direction detection protruding portions 221, 222, 223, and 224 may be made variable, according to the user preference for example, by placing a cap or the like.

In the foot position detecting device 200 according to the embodiment, as illustrated in FIG. 6A, the direction detection protruding portions 221, 222, 223, and 224 are provided at both ends on each of diagonal lines of the upper surface of the foot position detecting device 200 having a quadrangular shape as a whole. In other words, a direction obtained by inclining, by 45 degrees, a straight line connecting midpoints of opposed sides of the quadrangular upper surface of the foot position detecting device is set as a zero-degree reference direction. Hence, as also described above, a straight line connecting the direction detection protruding portion 221 and the direction detection protruding portion 223 to each other is set as the Y-axis, and a straight line connecting the direction detection protruding portion 222 and the direction detection protruding portion 224 to each other is set as the X-axis.

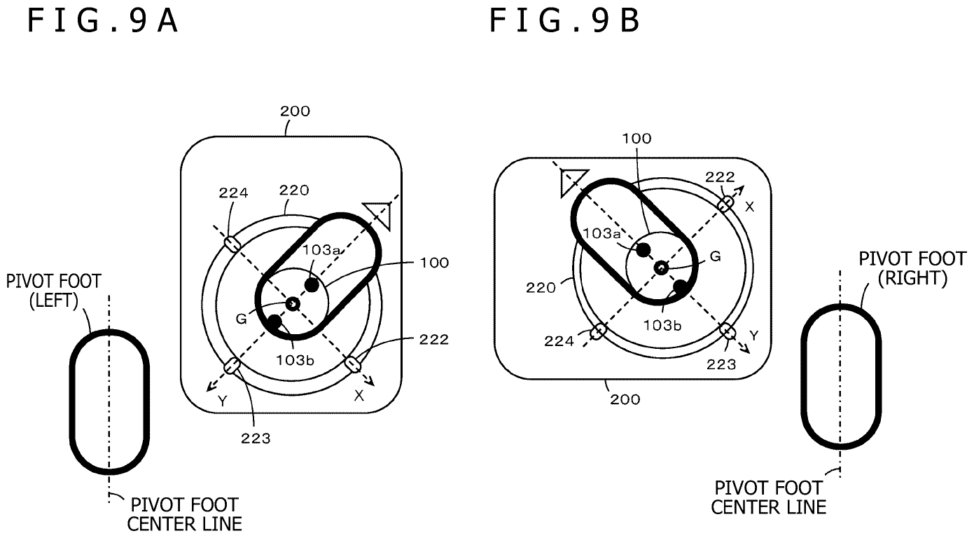

Thus, the user can reliably perform a rotating operation using either of the left and right feet as a pivot foot, and using the other foot as an indicating foot fitted with the foot position indicating instrument 100. FIGS. 9A and 9B are diagrams of assistance in explaining positional relation between the foot position detecting device 200, the pivot foot of the user, and the indicating foot of the user fitted with the foot position indicating instrument 100. Of FIGS. 9A and 9B, FIG. 9A represents an example in a case where a left foot is set as the pivot foot, and FIG. 9B represents an example in a case where a right foot is set as the pivot foot.

As illustrated in FIG. 9A, in the case where the left foot is set as the pivot foot, the foot position detecting device 200 is disposed such that a direction inclined to the right by 45 degrees with respect to a center line of the left foot (pivot foot center line) is the same direction as the Y-axis direction of the foot position detecting device 200. As also described above, the Y-axis direction of the foot position detecting device 200 is the same direction as the extending direction of the straight line connecting the direction detection protruding portion 221 and the direction detection protruding portion 223 to each other. Thus, as illustrated in FIG. 9A, the pivot foot (left foot) can be positioned so as to be parallel with the long sides of the foot position detecting device 200. The indicating foot can therefore be turned in either of a right direction and a left direction. Hence, the foot position indicating instrument 100 can be rotated in an intended direction easily and precisely.

In addition, as illustrated in FIG. 9B, in the case where the right foot is set as the pivot foot, the foot position detecting device 200 is disposed such that a direction inclined to the left by 45 degrees with respect to a center line of the right foot (pivot foot center line) is the same direction as the Y-axis direction of the foot position detecting device 200. As also described above, the Y-axis direction of the foot position detecting device 200 is the same direction as the extending direction of the straight line connecting the direction detection protruding portion 221 and the direction detection protruding portion 223 to each other. Thus, as illustrated in FIG. 9B, the pivot foot (right foot) can be positioned in parallel with the short sides of the foot position detecting device 200. The indicating foot can therefore be turned in either of the right direction and the left direction. Hence, the foot position indicating instrument 100 can be rotated in an intended direction easily and precisely.

Thus, in a case where the pivot foot side to which the foot position indicating instrument 100 is not attached is placed in parallel with the foot position detecting device 200, the indicating foot side to which the foot position indicating instrument 100 is attached is placed in a state of being opened to the outside by 45 degrees. The indicating foot to which the foot position indicating instrument 100 is attached is turned in either of the left and right directions easily. Hence, the foot position indicating instrument 100 fitted to the foot portion of the user can be rotated easily in either of the left and right directions.

Control of Output from Foot Input System

In the foot input system according to the present embodiment, the position indicated by the foot position indicating instrument 100 and the rotational angle thereof can be detected as those in an absolute coordinate system, or detected as those in a relative coordinate system. In the following, the absolute coordinate system that can be used in the foot input system according to the present embodiment will first be described, and thereafter the relative coordinate system that can be used in the foot input system according to the present embodiment will be described.

### Setting of Absolute Coordinate System

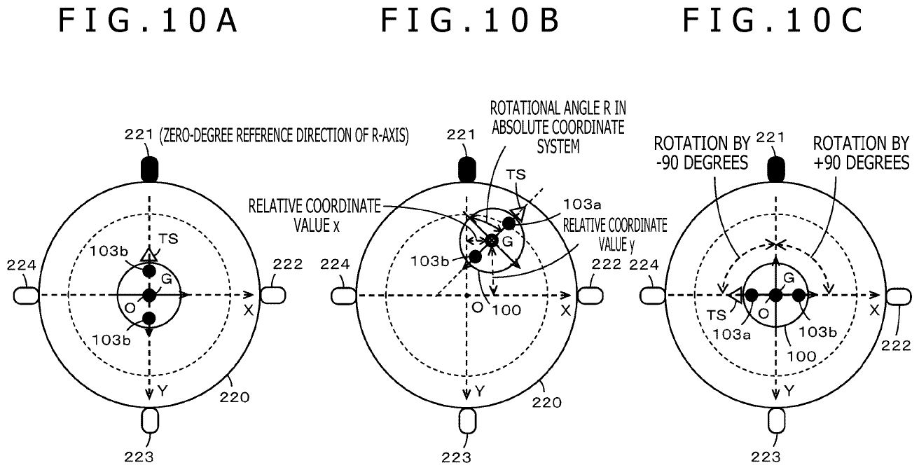

FIGS. 10A to 10C are diagrams of assistance in explaining the absolute coordinate system that can be used in the foot input system according to the present embodiment constituted by the foot position indicating instrument 100 and the foot position detecting device 200. In the foot input system according to the present embodiment, as also described above, the operation surface used by the foot position indicating instrument 100 is formed on the position detecting unit 220 of the foot position detecting device 200. As described with reference to FIGS. 6A and 6B, the position detecting unit 220 has the direction detection protruding portions 221, 222, 223, and 224 provided at intervals of 90 degrees so as to be laid on the outer wall-shaped protruding portion 220c of the position detecting unit 220, that is, on the outer edge of the position detecting unit 220.

In the case of the absolute coordinate system, as illustrated in FIGS. 10A, 10B, and 10C, the straight line connecting together the center of the direction detection protruding portion 221 and the center of the direction detection protruding portion 223 that are provided to the position detecting unit 220 is the Y-axis, and the straight line connecting together the center of the direction detection protruding portion 224 and the center of the direction detection protruding portion 222 is the X-axis. The Y-axis and the X-axis remain unchanged on the position detecting unit 220, and can therefore be referred to as an absolute Y-axis and an absolute X-axis. Moreover, as illustrated in FIGS. 10A, 10B, and 10C, a point of intersection of the X-axis (axis of abscissas) and the Y-axis (axis of ordinates) is an origin O (0, 0) on the position detecting unit 220.

The midpoint G as the middle position of the straight line connecting the centers of the coil 103a and the coil 103b of the foot position indicating instrument 100 to each other is the rotational axis of the foot position indicating instrument 100 (R-axis). In this case, the Y-axis is a 0 (zero) degree reference of the rotational axis (R-axis) of the foot position indicating instrument 100 on the position detecting unit 220. In FIGS. 10A, 10B, and 10C, a triangle TS illustrated in the vicinity of the foot position indicating instrument 100 indicates a direction in which a toe of the foot portion of the user fitted with the foot position indicating instrument is directed (toe direction). Specifically, a direction in which an apex of the triangle TS present on the straight line connecting the centers of the coil 103a and the coil 103b points to is the toe direction. It is therefore possible to grasp the rotational angle of the foot position indicating instrument 100 on the basis of how much the straight line connecting together the center of the coil 103a and the center of the coil 103b of the foot position indicating instrument 100 is inclined with respect to the Y-axis according to a change in the toe direction.

Hence, as illustrated in FIG. 10A, in the foot position detecting device 200, the top of the position detecting unit 220 is treated as the absolute coordinate system in which the point of intersection of the X-axis and the Y-axis is set as the origin O (0, 0) and the zero-degree reference direction of the rotational axis (R-axis) of the foot position indicating instrument 100 is set as the direction of the direction detection protruding portion 221 on the Y-axis. Thus, the foot input system including the foot position indicating instrument 100 and the foot position detecting device 200 can change each of a value on the X-axis, a value on the Y-axis, and the rotational angle on the R-axis in the absolute coordinate system in a manner similar to the analog axes of a joystick, for example. The foot input system according to the present embodiment can therefore be used as a controller for a computer game.

More specifically, as illustrated in FIG. 10B, when the foot position indicating instrument 100 is moved in the X-axis direction and the Y-axis direction on the position detecting unit 220, coordinate values (an absolute coordinate value X and an absolute coordinate value Y) corresponding to the position of the midpoint G of the foot position indicating instrument 100 on the position detecting unit 220 are output. In addition, when the foot fitted with the foot position indicating instrument 100 is turned on the position detecting unit 220, a rotational angle R (rotational angle R in the absolute coordinate system) corresponding to a direction in which the foot position indicating instrument 100 is directed is output. Incidentally, as illustrated in FIG. 10C, the rotational angle R in the absolute coordinate system can be detected in a range of −90 degrees (minimum value) to +90 degrees (maximum value).

Hence, as described with reference to FIGS. 10A to 10C, the X-axis, the Y-axis, and the rotational axis (R-axis) in the absolute coordinate system of the foot input system are assigned to the analog axes of forward/rearward and left/right movements and a rotational movement of an ordinary game controller, for example. Thus, the foot input system allows forward/rearward and left/right movements and a rotational movement to be performed in a manner similar to the game controller. That is, the foot input system can be used as the game controller. Incidentally, in FIGS. 10A, 10B, and 10C, a dotted line circle indicates a maximum distance between the origin O (0, 0) of the position detecting unit 220 and the midpoint (center of gravity) G of the foot position indicating instrument 100. This is because the outer edge of the position detecting unit 220 is provided with the outer wall-shaped protruding portion 220c, and the outer edge of the foot position indicating instrument 100 normally abuts against the outer wall-shaped protruding portion 220c, so that the foot position indicating instrument 100 cannot move outward of the outer wall-shaped protruding portion 220c.

### Provision Against VR Sickness

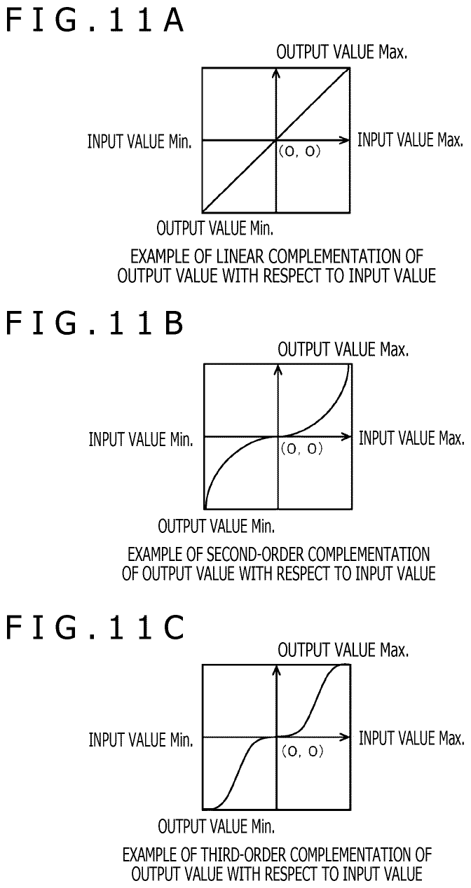

FIGS. 11A to 11C, FIG. 12, and FIGS. 13A to 13C are diagrams of assistance in explaining output value complementation for an input value. As for change amounts of input values with respect to the X-axis, the Y-axis, and the R-axis (rotational axis of the foot position indicating instrument 100) in the absolute coordinate system on the position detecting unit 220, it is not desirable to detect a movement of the foot position indicating instrument 100 as is relative to the origin position and the reference direction. That is, it is not desirable to detect the change amounts (output values) of the values with respect to the X-axis, the Y-axis, and the R-axis linearly in proportion to the movement (input values) of the foot position indicating instrument 100, as illustrated in FIG. 11A.

The foot input system becomes easier to use when the change amounts (output values) of the values with respect to the X-axis, the Y-axis, and the R-axis, which correspond to the movement (input values) of the foot position indicating instrument 100, are detected as nonlinear changes. This has an effect of suppressing an unintended input operation, to thereby reduce stress caused by VR sickness. Incidentally, VR sickness (Virtual Reality Sickness) is a phenomenon (symptom) of dizziness, nausea, discomfort, or the like caused at a time of viewing VR video while wearing VR goggles or undergoing a metaverse experience, and is a symptom very similar to “motion sickness.”

Specifically, the foot input system according to the present embodiment obtains an output value by performing predetermined complementation processing on an input value as follows. As for output values on the X-axis and the Y-axis corresponding to forward/rearward or left/right movements of the foot position indicating instrument 100, for example, the output values are changed to values obtained by subjecting the respective input values to second-order complementation, that is, values obtained by squaring and normalizing an input value Xin or an input value Yin, as illustrated in FIG. 11B. In addition, as for the rotational angle R in a case of a rotational movement of the foot position indicating instrument 100 with the midpoint G as the rotational axis, the output value is changed to a value obtained by third-order complementation, that is, a value obtained by normalizing −2Rin+3Rin2 for an input value Rin, as illustrated in FIG. 11C.

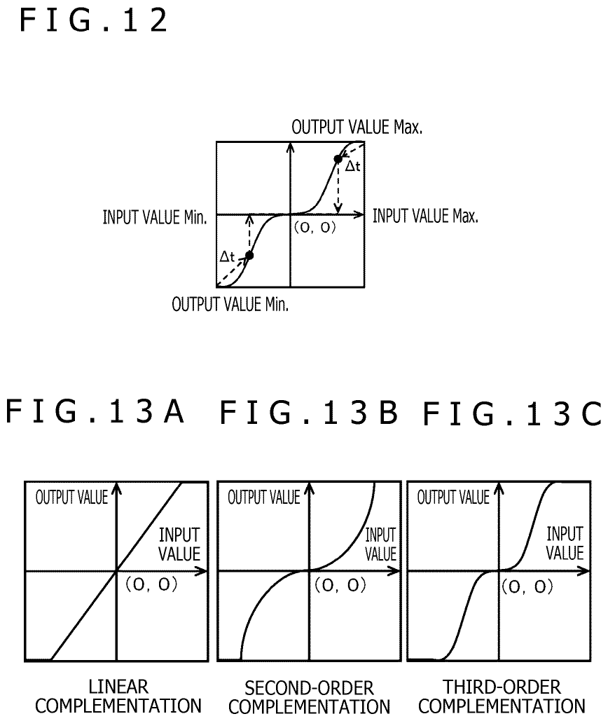

Consequently, a fine movement operation and a quick movement operation are well supported by the foot input system. Hysteresis may be provided to an output value with respect to an input. For example, in a case of the rotational angle R (R-axis) of a rotational movement, in particular, it is preferable to stop the rotational movement immediately when the rotation of a screen reaches a target angle, also as a measure against VR sickness. Accordingly, as illustrated in FIG. 12, after a rotational movement is performed by the rotational angle R, the output value of the rotational angle R (R-axis) is set to be zero immediately when an operation of returning to the orientation in the reference direction is performed quickly, as indicated by Δt. It is thereby possible to stop the rotation of the screen immediately even when the R-axis has not returned to the reference direction.

The degree of such complementation is not set to be one specific degree, but may be optionally adjustable. For example, as illustrated in FIGS. 13A, 13B, and 13C, complementation may be performed such that the output value is set at a maximum before the input value assumes a maximum. In addition, whether to perform second-order complementation, third-order complementation, or linear complementation proportional to the input values for the X-axis, the Y-axis, and the R-axis may be suitably determined. In addition, the degree of complementation may be varied between a forward movement operation and a rearward movement operation, that is, the positive side and the negative side of the input value.

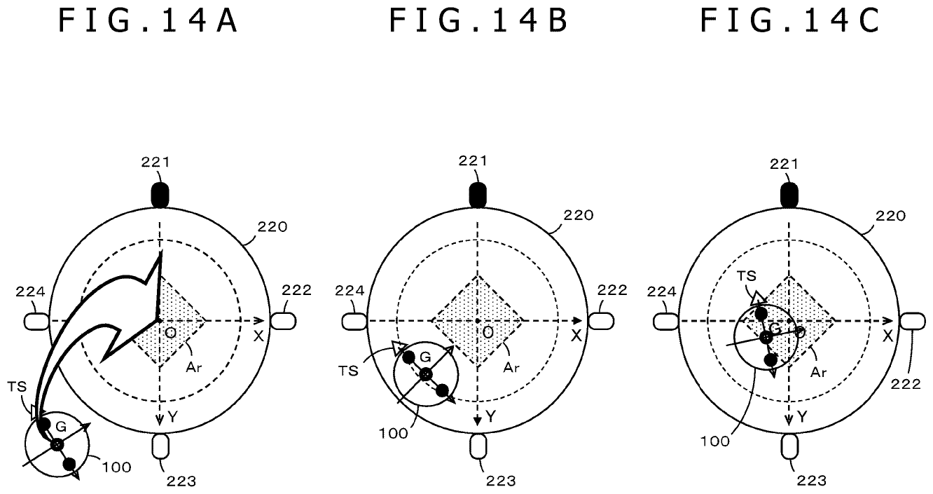

Further, the foot position indicating instrument 100 may be moved to the outside of the casing of the foot position detecting device 200 (outside of the position detecting unit 220) during an operation of the foot position indicating instrument 100 on the position detecting unit 220 of the foot position detecting device 200. In that case, all of the output values become zero values, and thus a state is set in which no input processing is performed. FIGS. 14A to 14C are diagrams of assistance in explaining a case where the foot position indicating instrument 100 is moved to the outside of the position detecting unit 220. As illustrated in FIG. 14A, suppose that the foot position indicating instrument 100 is moved to the outside of the position detecting unit 220 of the foot position detecting device 200.

Consideration will be given to a case where the foot position indicating instrument 100 is thereafter returned to the inside of the position detecting unit 220 of the foot position detecting device 200. In the case where the return is made, it is desirable to make the return to the inside of a predetermined area (a return-determination threshold value range) Ar indicated by a quadrangle having the origin O (0, 0) as a center thereof in FIG. 14A in order to prevent unintentional output of large movement values. However, suppose that at the time of the return, as illustrated in FIG. 14B, the midpoint (center of gravity) G and the toe direction of the foot position indicating instrument 100 greatly deviate from the origin O (0, 0) and the direction of the direction detection protruding portion 221 on the Y-axis. That is, suppose that the return position of the foot position indicating instrument 100 greatly deviates from the return-determination threshold value range Ar. In this case, output processing for the input values of the foot position indicating instrument 100 is not performed, and the output values are treated as X=0, Y=0, and R=0.

Thereafter, the output processing for the input values is resumed when the return position of the midpoint G of the foot position indicating instrument 100 is located within the return-determination threshold value range Ar, as illustrated in FIG. 14C. That is, the foot position detecting device 200 resumes the processing of outputting a position (X, Y) indicated by the foot position indicating instrument 100 and the rotational angle R of the foot position indicating instrument 100 according to an indication by the foot position indicating instrument 100. It is thereby possible to prevent unintended production of a high movement output and to suppress VR sickness caused by unintended movement of the screen when returning the foot position indicating instrument 100 to the inside of the position detecting unit 220. Incidentally, control on the absolute coordinate system described with reference to FIGS. 10A to 10C allows an operation to be performed in all directions not only in a case where the user fitted with the foot position indicating instrument 100 performs the operation in a state of a standing position but also in a case where the user fitted with the foot position indicating instrument 100 performs the operation in a state of a sitting position, which is a state of being seated in a chair or the like.

### Setting of Relative Coordinate System

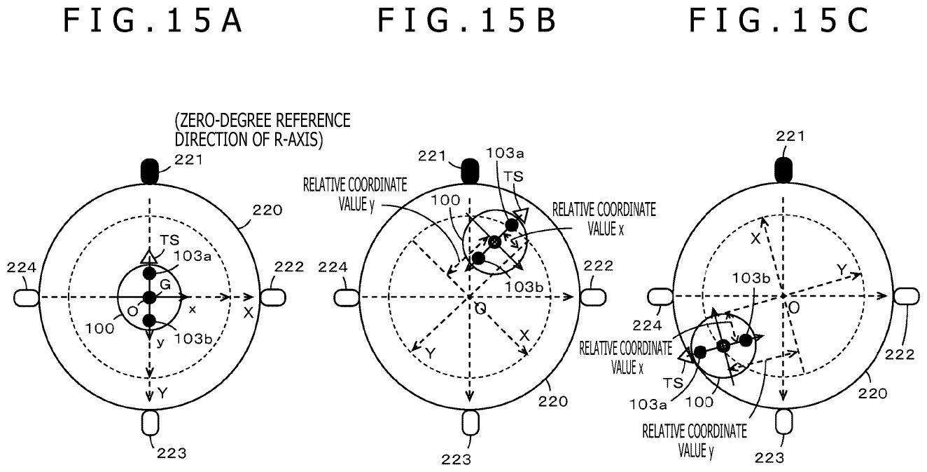

FIGS. 15A to 15C are diagrams of assistance in explaining the relative coordinate system that can be used in the foot input system according to the present embodiment constituted by the foot position indicating instrument 100 and the foot position detecting device 200. In the case of the relative coordinate system, the X-axis and the Y-axis are not fixedly provided, but instead the direction in which the toc of the foot of the user fitted with the foot position indicating instrument 100 is directed is always the reference direction. That is, the position detecting unit 220 has the relative coordinate system in which the direction in which the toe of the foot of the user fitted with the foot position indicating instrument 100 is directed is a relative Y-axis, which is a forward/rearward movement direction, and an axis orthogonal to the relative Y-axis is set as a relative X-axis, which is a left/right movement direction. In this example, the R-axis (rotational angle R) is used in the calculation of the relative coordinate system and, thus, not possessed as an output value based on the angle information of the foot position indicating instrument. In other words, the output of a rotation indication is not performed in the present example.

The relative coordinate system will be specifically described. As illustrated in FIGS. 15A, 15B, and 15C, in the foot input system according to the present embodiment, as also described above, the operation surface used by the foot position indicating instrument 100 is formed on the position detecting unit 220 of the foot position detecting device 200. As described with reference to FIGS. 6A and 6B, the position detecting unit 220 has the direction detection protruding portions 221, 222, 223, and 224 provided at intervals of 90 degrees so as to be laid on the outer wall-shaped protruding portion 220c of the position detecting unit 220, that is, on the outer edge of the position detecting unit 220. Also in the relative coordinate system, as in the case of the absolute coordinate system described with reference to FIGS. 10A to 10C, a point of intersection of the straight line connecting the direction detection protruding portion 221 and the direction detection protruding portion 223 to each other and the straight line connecting the direction detection protruding portion 222 and the direction detection protruding portion 224 to each other is the origin O (0, 0).

Also in FIGS. 15A, 15B, and 15C, the foot position indicating instrument 100 is illustrated as a small circle, within which the coils 103a and 103b are illustrated as black circles, and the middle position of the straight line connecting the centers of the coil 103a and the coil 103b to each other is illustrated as the midpoint (center of gravity) G. In addition, a triangle TS illustrated in the vicinity of the foot position indicating instrument 100 indicates a direction in which the toe of the foot portion of the user fitted with the foot position indicating instrument is directed (toe direction). Specifically, a direction in which an apex of the triangle TS present on the straight line connecting the centers of the coil 103a and the coil 103b points to is the toc direction. Incidentally, in FIGS. 15A, 15B, and 15C, as in the case of FIGS. 10A, 10B, and 10C, a dotted line circle indicates a maximum distance between the origin O (0, 0) of the position detecting unit 220 and the midpoint (center of gravity) G of the foot position indicating instrument 100.

In FIGS. 15A, 15B, and 15C, the straight line connecting the centers of the coil 103a and the coil 103b of the foot position indicating instrument 100 to each other is a y-axis on the indicating instrument, and a straight line that passes through the midpoint G of the foot position indicating instrument 100 and is orthogonal to the y-axis on the indicating instrument is an x-axis on the indicating instrument. In addition, a straight line that is parallel with the y-axis on the indicating instrument and passes through the origin O is a relative reference Y-axis on the position detecting unit 220, and a straight line that is parallel with the x-axis on the indicating instrument and passes through the origin O is a relative reference X-axis on the position detecting unit 220.

As illustrated in FIG. 15A, suppose that the midpoint G of the foot position indicating instrument 100 is located on the origin O of the position detecting unit 220, that the y-axis on the indicating instrument coincides with the relative reference Y-axis, and that the x-axis on the indicating instrument coincides with the relative reference X-axis. In this case, the foot position detecting device 200 detects the position of the midpoint G of the foot position indicating instrument 100 as an indicated position, and therefore the indicated position can be detected as a position coinciding with the origin O (0, 0). Incidentally, in the case of the example illustrated in FIG. 15A, the toc direction of the user fitted with the foot position indicating instrument 100 is a direction in which the direction detection protruding portion 221 is located. Therefore, a forward movement is indicated when the foot position indicating instrument 100 is moved in the direction of the direction detection protruding portion 221, and a rearward movement is indicated when the foot position indicating instrument 100 is moved in the direction of the direction detection protruding portion 223.

As illustrated in FIG. 15B, suppose that the foot position indicating instrument 100 is moved to an upper right part on the position detecting unit 220 and that the toe direction of the foot of the user fitted with the foot position indicating instrument 100 is a rightward and obliquely upward direction. In this case, as illustrated in FIG. 15B, the relative reference Y-axis and the relative reference X-axis are rotated in a right-handed direction (clockwise) from those illustrated in FIG. 15A in correspondence with the rotation of the foot position indicating instrument 100. In this case, as illustrated in FIG. 15B, an indicated position corresponding to the midpoint G of the foot position indicating instrument 100 can be identified by a relative coordinate value x and a relative coordinate value y on the basis of the relative reference X-axis and the relative reference Y-axis.

In addition, as illustrated in FIG. 15C, suppose that the foot position indicating instrument 100 is moved to a lower left side part on the position detecting unit 220 and that the toc direction of the foot of the user fitted with the foot position indicating instrument 100 is a leftward and obliquely downward direction. In this case, as illustrated in FIG. 15C, the relative reference Y-axis and the relative reference X-axis are rotated in a left-handed direction (counterclockwise) from those illustrated in FIG. 15A in correspondence with the rotation of the foot position indicating instrument 100. In this case, as illustrated in FIG. 15C, an indicated position corresponding to the midpoint G of the foot position indicating instrument 100 can be identified by the relative coordinate value x and the relative coordinate value y on the basis of the relative reference X-axis and the relative reference Y-axis.

Thus, the foot input system including the foot position indicating instrument 100 and the foot position detecting device 200 can change each of a value on the X-axis and a value on the Y-axis in the relative coordinate system in a manner similar to the analog axes of a joystick, for example. The foot input system according to the present embodiment can therefore be used as a controller for a computer game. That is, when the foot position indicating instrument 100 is moved in the relative reference X-axis direction and the relative reference Y-axis direction, values corresponding to the coordinate values of the foot position indicating instrument 100 are output. Thus, by assigning the axes to the analog axes of forward/rearward and left/right movements of an ordinary game controller, for example, it is possible to perform forward/rearward and left/right movements in a manner similar to those of the game controller.

In addition, also in the case of using the relative coordinate system, as in the case of using the absolute coordinate system described above, the relative coordinate value x and the relative coordinate value y are not limited to being changed linearly in proportion with respect to the origin position, but may be changed nonlinearly. It is thereby possible to provide improved usability, and to reduce stress caused by so-called VR sickness. For example, in a case of the relative X-axis corresponding to a forward/rearward movement and the relative Y-axis corresponding to a left/right movement, the output values are changed by values obtained by subjecting the respective input values (detected values) to quadratic function complementation, that is, values obtained by squaring and normalizing a relative input value x or a relative input value y. Consequently, a fine movement operation and a quick movement operation are supported. Incidentally, the relative input value x means an input (detected) relative coordinate value x, and the relative input value y means an input (detected) relative coordinate value y.

In addition, hysteresis may be provided to an output value with respect to an input value. The degree of the complementation is not set to be one specific degree, but may be optionally adjustable. In addition, whether to perform quadratic function complementation, cubic function complementation, or linear output proportional to the input values for the relative input value x and the relative input value y may be suitably set and adjusted. In addition, the degree of the complementation may be changed between a forward movement operation and a rearward movement operation. In addition, the foot position indicating instrument 100 may be moved to the outside of the position detecting unit 220 of the foot position detecting device 200 during an operation of the foot position indicating instrument 100 on the position detecting unit 220 of the foot position detecting device 200. In that case, all of the output values become zero values, and a state is set in which no input processing is performed.

When the foot position indicating instrument 100 is returned onto the position detecting unit 220 of the foot position detecting device 200, processing is performed in a similar manner to the case of using the absolute coordinate system described with reference to FIGS. 14A to 14C. Specifically, suppose that the position of the foot position indicating instrument 100 greatly deviates from the origin O (0, 0) on the position detecting unit 220 when the foot position indicating instrument 100 is returned onto the position detecting unit 220 of the foot position detecting device 200. In this case, as described with reference to FIGS. 14A to 14C, coordinate values (relative values) with respect to the relative reference X-axis and the relative reference Y-axis are output only after the foot position indicating instrument 100 is moved to the inside of the predetermined area (the return-determination threshold value range) Ar in the vicinity of the origin O (0, 0). It is thereby possible to prevent unintended production of a high movement output to thereby suppress VR sickness caused by unintended movement of the screen, when the foot position indicating instrument 100 is returned onto the position detecting unit 220 of the foot position detecting device 200.

### Single Axis Control (Relative Y-Axis Control) Using Relative Coordinate System

As described with reference to FIGS. 15A to 15C, in the case of the relative coordinate system, a forward/rearward movement and a left/right movement can be indicated by using the foot position indicating instrument 100 even when the toe direction of the foot of the user fitted with the foot position indicating instrument 100 on the position detecting unit 220 is directed in any direction. It is therefore possible to perform control in the foot input system such that only the forward/rearward movement, for example, can be indicated by using the foot position indicating instrument 100. In this case, the foot position detecting device 200 detects only the relative coordinate value y as a value to be detected according to the movement of the foot position indicating instrument 100 on the position detecting unit 220, and the relative coordinate value x is set at 0 (zero) at all times.

Control using only the relative coordinate value y (relative Y-axis control) enables the output value of a forward/rearward movement to be adjusted by turning the foot fitted with the foot position indicating instrument 100, that is, by changing the angle of the foot position indicating instrument 100. In the cases of control using the absolute coordinate system described with reference to FIGS. 10A to 10C and control using the relative coordinate system described with reference to FIGS. 15A to 15C, the output values on the X-axis and the Y-axis can be brought close to zero by bringing the foot position indicating instrument 100 close to the origin O (0, 0).

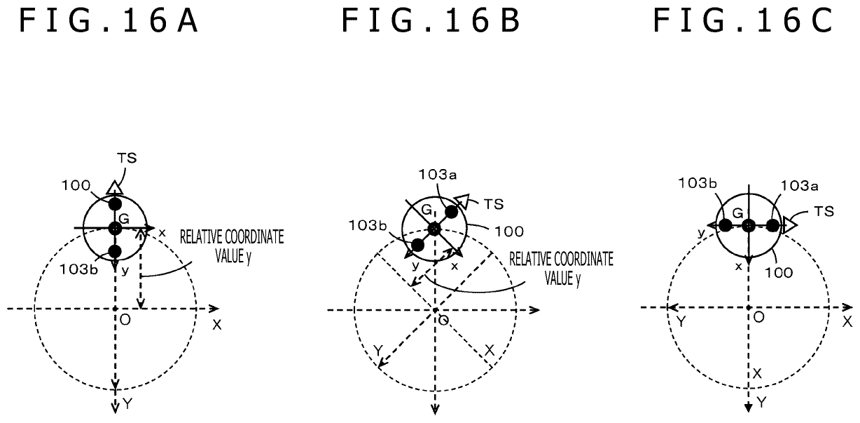

In the case of the control using only the relative coordinate value y (relative Y-axis control), on the other hand, the relative coordinate position y can be changed by merely turning the foot fitted with the foot position indicating instrument 100. FIGS. 16A to 16C are diagrams of assistance in explaining the control using only the relative coordinate value y (relative Y-axis control). As illustrated in FIG. 16A, suppose that the foot position indicating instrument 100 is moved on the relative reference Y-axis, and that the midpoint (center of gravity) G of the foot position indicating instrument 100 reaches the maximum distance from the origin O (0, 0) of the position detecting unit 220 indicated by a dotted line circle.

In this case, the relative coordinate value y assumes a minimum value or a maximum value according to the direction of a sign of the relative reference Y-axis. That is, when the midpoint G of the foot position indicating instrument 100 is moved on the relative reference Y-axis and reaches the circle indicated by a dotted line, a distance from the origin O (0, 0) to the midpoint G is longest. Hence, in this case, as indicated by the double-headed arrow in FIG. 16A, the relative coordinate value y assumes a minimum or a maximum. Incidentally, the minimum or the maximum is assumed because, in FIG. 16A, the position of the midpoint G is the minimum in a case where the relative reference Y-axis is positive in a downward direction, and the position of the midpoint G is the maximum in a case where the relative reference Y-axis is positive in an upward direction.

When the foot fitted with the foot position indicating instrument 100 is turned as illustrated in FIG. 16B from the state illustrated in FIG. 16A, the relative coordinate system using the foot position indicating instrument 100 as a reference is rotated, and the value of the relative coordinate value y approaches zero. That is, because the relative reference X-axis approaches the original relative reference Y-axis as the foot position indicating instrument 100 is rotated, a distance between the midpoint G of the foot position indicating instrument 100 and the relative reference X-axis becomes increasingly shorter, and the value of the relative coordinate value y becomes increasingly smaller, as indicated by the double-headed arrow in FIG. 16B.

As illustrated in FIG. 16C, when the relative reference X-axis completely coincides with the original relative reference Y-axis, the relative coordinate value y becomes 0 (zero). When the foot position indicating instrument 100 is further rotated beyond this point in the same rotational direction, the sign of the value of the relative coordinate value y is reversed. When the relative reference Y-axis of the relative coordinate system eventually crosses the origin O (0, 0) on the position detecting unit 220 of the foot position detecting device 200, the value of the relative coordinate value y becomes a maximum value or a minimum value. Incidentally, in the case of the example illustrated in FIGS. 16A to 16C, it is assumed that the relative coordinate value y changes, but the relative coordinate value x is 0 (zero) at all times, as described above.

Such configuration described above makes possible to perform a decelerating operation naturally similar to, for example, when the user changes the angle of the body to make a turn at a right angle in a real life. Therefore, it becomes possible to omit an operation of returning the foot position indicating instrument 100 to the center on the position detecting unit 220 of the foot position detecting device 200, to thereby allow for intuitive movement within VR which avoids VR sickness caused by unnatural video movement.

Incidentally, description using FIGS. 16A to 16C has been made by taking as an example a case where the midpoint of the foot position indicating instrument 100 is moved to a position at which a maximum value or a minimum value is assumed, and then the foot position indicating instrument 100 is rotated, in order to simplify the description. However, the midpoint of the foot position indicating instrument 100 does not necessarily need to be moved to a position at which a maximum value or a minimum value is assumed. The single axis control using the relative coordinate system (relative Y-axis control) can be performed as described with reference to FIGS. 16A to 16C irrespective of the position of the foot position indicating instrument 100 on the position detecting unit 220.

Thus, in the case of the foot input system according to the present embodiment, three types of coordinate system control can be used from the values of the position and angle of the foot position indicating instrument 100 used on the position detecting unit 220 of the foot position detecting device 200. Specifically, it is possible to perform three types of basic control, that is, three-axis control on the X-axis, the Y-axis, and the R (rotation) axis of the absolute coordinate system (FIGS. 10A to 10C), two-axis control on the X-axis and the Y-axis of the relative coordinate system (FIGS. 15A to 15C), and the single axis control on the Y-axis of the relative coordinate system. The three types of basic control can be suitably selected according to applications. For example, it suffices to mount the foot position detecting device 200 with a push button for control switching so that a function (e.g., the type of coordinate system control) is switched each time the push button is depressed. It is thereby possible to change the coordinate system control to be used according to, for example, a computer game executed in the image processing device 500.

### Examples of Application of Coordinate System Control

The foot input system according to the embodiment described above can perform three types of basic control, that is, the three-axis control on the X-axis, the Y-axis, the R (rotation) axis of the absolute coordinate system (FIGS. 10A to 10C), the two-axis control on the X-axis and the Y-axis of the relative coordinate system (FIGS. 15A to 15C), and the single axis control on the Y-axis of the relative coordinate system. The following describes the image processing system in cases where these different types of coordinate system control are applied.

### Example of Application of Single Axis Control (Relative Y-Axis Control) Using Relative Coordinate System

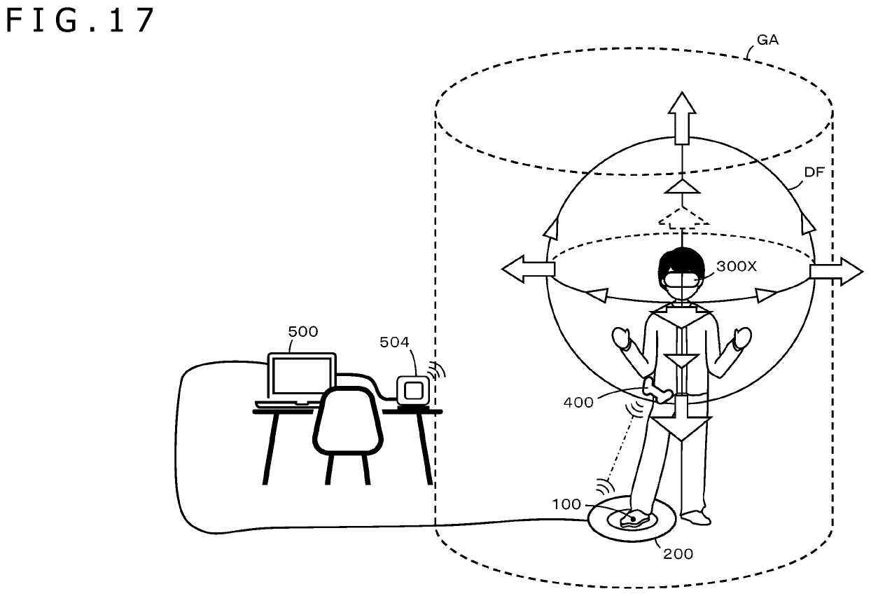

FIG. 17 is a diagram of assistance in explaining a concrete example of the single axis control (relative Y-axis control) using the relative coordinate system, and is, more specifically, a diagram of assistance in explaining the operation state of the foot input system based on the relative Y-axis control and VR application using an HMD 300X. Hence, in the image processing system illustrated in FIG. 17, the foot input system including the foot position indicating instrument 100 and the foot position detecting device 200 receives an indication input of only the forward/rearward movement. In the image processing system illustrated in FIG. 17, the foot position detecting device 200 is connected via wire to a PC 500 as an image processing device, and the HMD 300X is connected to the PC 500 wirelessly through a communicating unit 504 such as a Wi-Fi® router. That is, the communicating unit 504 corresponds to the communicating unit 504 of the image processing device 500 illustrated in FIG. 2.

In addition, suppose that a hand operation within VR is performed by hand tracking. A hand tracking technology is typically a technology in which a camera attached to the HMD 300X recognizes a hand of a person fitted with the HMD 300X, and the position of the hand is reflected in a hand of an avatar in the VR space. Hence, in FIG. 17, while the HMD 300X has an external appearance similar to that of the HMD 300 described above, the HMD 300X has a camera function in addition to the processing of displaying stereoscopic video, and performs tracking and input processing of the positions and angles of the HMD 300X and both hands and further performs tracking and input processing of how the fingers are bent.

Incidentally, in the image processing system illustrated in FIG. 17, a hand operation within VR is performed by hand tracking, and therefore the game controller 400 does not need to be used. However, the game controller 400 can wirelessly communicate with the foot position detecting device 200, and is connected to the PC 500 through the foot position detecting device 200. Alternatively, as with the HMD 300, the game controller 400 can also be wirelessly connected to the PC 500 through a wireless transceiver. It is thereby possible to configure the system as a system similar to the image processing system illustrated in FIG. 1.

In the present example, a 360-degree image region GA indicated by a cylindrical body of a dotted line to surround the user corresponds to a range reachable by the hands of the user when the user moves about the foot position detecting device 200. In other words, the 360-degree image region GA corresponds to a range in which there is a possibility of the user physically colliding with an object or a wall during the usage of the image processing system including the foot input system according to the present embodiment. In addition, a range indicated by a spherical body DF covering an upper body of the user and a space over the head of the user represents a VR space, which supports a linear movement and a rotational movement the three axes including an X-axis, a Y-axis, and a Z-axis. That is, the spherical body DF is a so-called 6DoF (Degree of Freedom) VR space that supports not only the “rotation and inclination” but also forward/rearward, left/right, and upward/downward movements of the head and the neck.

In general, in a case where an operation for movement within VR is performed by using an ordinary input device such as a joystick held in a hand, discomfort similar to motion sickness may occur because the movement of a field of view and the inertia of a bodily sensation do not match each other. However, in the case of the image processing system formed using the foot input system illustrated in FIG. 17, the movement of the field of view and the inertia of the bodily sensation match each other in a rotational movement which particularly tends to trigger VR sickness. It is therefore possible to suppress the occurrence of VR sickness. In addition, because the movement of putting the foot forward or rearward effectively prepares the body as a whole for the movement, VR sickness can be reduced in the forward/rearward movement also.

In the case of the image processing system illustrated in FIG. 17, rotation of a VR image appearing on the HMD 300X is performed only by the tracking of the HMD 300X. That is, the VR image is rotated according to the orientation of the HMD 300X. In a case where the user is present within the VR space, the field of view of the user is blocked by the HMD 300X, and therefore the user cannot grasp where the user is located within a room. During usage in a standing state, the user's execution position may be gradually shifted, and entanglement with a cable or an unintended contact with a wall may occur.

However, in the case of the image processing system illustrated in FIG. 17, the foot input system including the foot position indicating instrument 100 and the foot position detecting device 200 confines the body of the user to a position on the position detecting unit 220 of the foot position detecting device 200. Therefore, in the case of the image processing system illustrated in FIG. 17, relative space saving can be achieved, the execution position is not shifted, and the risk of cable entanglement is reduced also due to its wireless configuration, such that the image processing system can be used in a safe manner.

With an ordinary VR controller, a large movement of the hands is used to indicate movement of the body and, thus, as compared with a conventional computer game or the like, operations and movements become much more complicated. On the other hand, in the case of the image processing system illustrated in FIG. 17, the user's hands are free and the user can freely grab an object within the VR space as in reality. Operations within the VR space can be performed in a manner corresponding to actual movements in reality. For example, a movement operation can be indicated by moving the foot forward or rearward, and a rotating operation can be indicated by rotating the user body. Thus, even a person unaccustomed to the computer can easily and intuitively perform an operation within the VR space.

In the case of the image processing system illustrated in FIG. 17, when the hand tracking technology is used, it is possible to perform an operation within the VR space without holding a VR controller, but it is not possible to perform a movement operation using a joystick or the like. A movable range is therefore limited to the physical area of a room. However, in the case of the image processing system illustrated in FIG. 17, the foot input system is used to support a movement operation, and it is therefore possible to perform a movement operation within the VR space while supporting the hand tracking such as to hold an object with both hands.

In addition, in a case of a conventional ordinary image processing system for forming a VR space, a game controller held in hands is used to perform a movement operation, which, in reality, is performed by a movement of walking using the user's feet. Therefore, the user cannot move the hands naturally, and a sense of immersion, which is an important characteristic of VR, is diminished. On the other hand, in the case of the image processing system described with reference to FIG. 17, the hands can be dedicated to the hand operation only, and the hand and finger movements can be freely reproduced and rendered within the VR space to achieve a sense of immersion as if the hands are present within the VR space.

There are existing movement operation devices using legs. However, the existing movement operation devices using legs require large-sized device bodies, cause fatigue due to repeated stepping movement required, and suffer from limited usable postures, an input delay, and a difficulty in performing a fine operation when only a slight movement is desired.

On the other hand, in the case of the image processing system illustrated in FIG. 17, the foot position detecting device 200 using the position detecting sensor of the electromagnetic induction type can perform position detection in a noncontact manner and on a battery-less basis. It is therefore possible to realize a small-sized and lightweight foot position detecting device 200 close to a B4 paper size. Thus, in the case of the image processing system illustrated in FIG. 17, an operation such as continuous stepping during movement is not necessary, various postures can be freely assumed because the body is not constrained, and no input delay is felt. In addition, in the case of the image processing system illustrated in FIG. 17, even a slight position change (a small amount of displacement) of the foot position indicating instrument 100 can be detected with high resolution and with high accuracy, so that a fine operation indication can be performed.

In the image processing system described with reference to FIG. 17, a hand operation within the VR space is performed by hand tracking, but the present disclosure is not so limited. A VR controller may be used, or a small wireless communication input terminal as a button input controller may be held in the hands or by a body part other than the hands. In addition, the HMD 300X and the PC 500 can be used in a state of being connected to each other via wire, and the foot position detecting device 200 and the PC 500 can be used in a state of being wirelessly connected to each other.

### Examples of X-Y-R-Axis Control Using Absolute Coordinate System

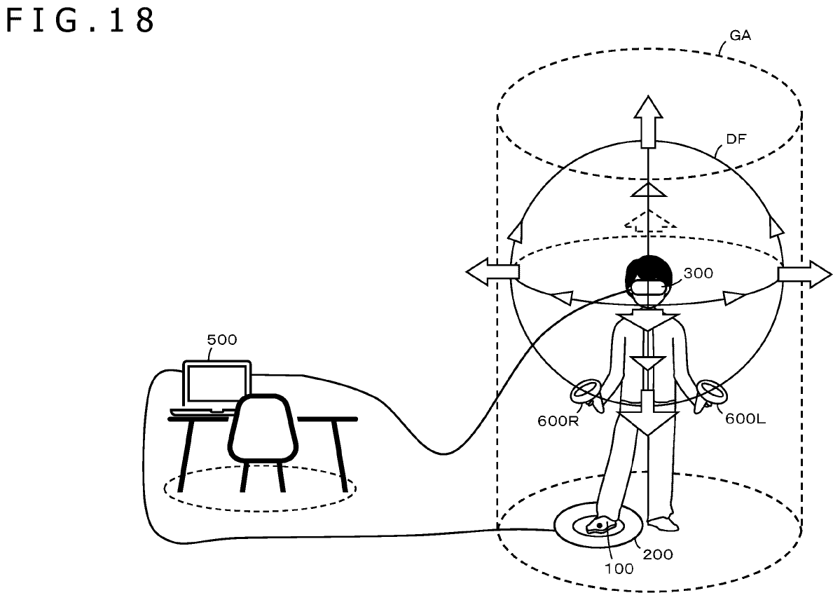

FIG. 18 is a diagram of assistance in explaining a concrete example of X-Y-R-axis control using the absolute coordinate system. More specifically, FIG. 18 is a diagram of assistance in explaining the operation state of the foot input system based on the X-Y-R-axis control using the absolute coordinate system and VR application using the HMD 300. In the example illustrated in FIG. 18, the foot position detecting device 200 is connected via wire to the PC 500 as an image processing device, and the HMD 300 is also connected to the PC 500 via wire. Hand operations within the VR space are performed by VR hand controllers 600L and 600R.

In FIG. 18, as in the case of the image processing system illustrated in FIG. 17, a 360-degree image region GA indicated by a cylindrical body of a dotted line to surround the user corresponds to a range reachable by the hands of the user when the user moves about the foot position detecting device 200. In other words, the 360-degree image region GA corresponds to a range in which there is a possibility of the user physically colliding with an object or a wall during the usage of the image processing system formed using the foot input system according to the present embodiment. In addition, a range indicated by a spherical body DF covering an upper body of the user and a space over the head of the user represents a VR space, which corresponds to a linear movement and a rotational movement on the three axes, that is, the X-axis, the Y-axis, and the Z-axis. That is, the spherical body DF is a so-called 6DoF (Degree of Freedom) VR space that handles not only the “rotation and inclination” of the head and the neck but also forward/rearward, left/right, and upward/downward movements of the head and the neck.

In general, in a case where operations for movements within VR are performed by using an ordinary input device such as a joystick held in a hand, discomfort similar to motion sickness may occur because the movement of the field of view and the inertia of the bodily sensation do not match each other. However, also in the case of the image processing system formed using the foot input system illustrated in FIG. 18, the movement of the field of view and the inertia of the bodily sensation match each other in a rotational movement which particularly tends to trigger VR sickness. It is therefore possible to suppress the occurrence of VR sickness. In addition, VR sickness can be reduced also in a forward/rearward movement because the movement of putting the foot forward or rearward effectively prepares the body as a whole for the movement, which causes movement of the whole body. Further, in the case of the image processing system illustrated in FIG. 18, the foot can be put not only forward or rearward but also leftward or rightward and can be rotated, to thereby effectively prepare the whole body for the movement. It is therefore possible to reduce VR sickness in the forward/rearward movement, in the left/right movement, and in the rotational movement.

Within the VR space, the field of view is blocked by the HMD 300, and therefore it is not possible to grasp where the user is located within a room. During usage in a standing state, the user's execution position may be gradually shifted, and entanglement with a cable or an unintended contact with a wall may occur. On the other hand, in the image processing system illustrated in FIG. 18, the foot input system including the foot position indicating instrument 100 and the foot position detecting device 200 confines the body of the user to a position on the position detecting unit 220 of the foot position detecting device 200. Therefore, also in the case of the image processing system illustrated in FIG. 18, relative space saving is achieved, the execution position is not shifted, and the risk of cable entanglement is reduced also due to its wireless configuration, such that the image processing system can be used safely.

In addition, as also described above, with an ordinary VR controller, a large movement of the hands is used to indicate movement of the body and, thus, as compared with a conventional computer game or the like, operations and movements become much more complicated. On the other hand, in the case of the image processing system illustrated in FIG. 18, the VR hand controllers 600L and 600R are used, but joysticks on the VR hand controllers are not used for a movement operation. A movement operation is performed by moving the foot forward or rearward or left or right, or turning the foot. It is therefore possible to perform an operation within the VR space wherein the hand movement does not cause confusion or interfere with the movement operation (using the foot).

In the image processing system illustrated in FIG. 18, a movement operation is performed by the foot through the foot position indicating instrument 100 and the foot position detecting device 200. On the other hand, in a conventional image processing system, a movement operation is performed by hand using a so-called game controller or the like. Therefore, the user cannot use the hands freely, and a sense of immersion, which is important characteristic of VR, is diminished. On the other hand, in the case of the image processing system illustrated in FIG. 18, the hands can be dedicated to the hand operation only, and the positions and directions of the hands are tracked and rendered within the VR space at high speed and with high accuracy, to achieve high performance operability and a sense of immersion.

In the image processing system illustrated in FIG. 18, a hand operation within the VR space is performed by using the VR controllers 600L and 600R. However, hand tracking may be used, or a small input terminal as a button input controller may be held in the hands or by a body part other than the hands. In addition, the HMD 300 and the PC 500 can be used in a state of being wirelessly connected to each other, and the foot position detecting device 200 and the PC 500 can be used in a state of being wirelessly connected to each other. The image processing system illustrated in FIG. 18 is based on absolute X-Y-R-axis control, and thus limits postures, but can be used also by the user seated in a chair.

### Usage of Stand-Alone VR Based on X-Y-R-Axis Control Using Absolute Coordinate System

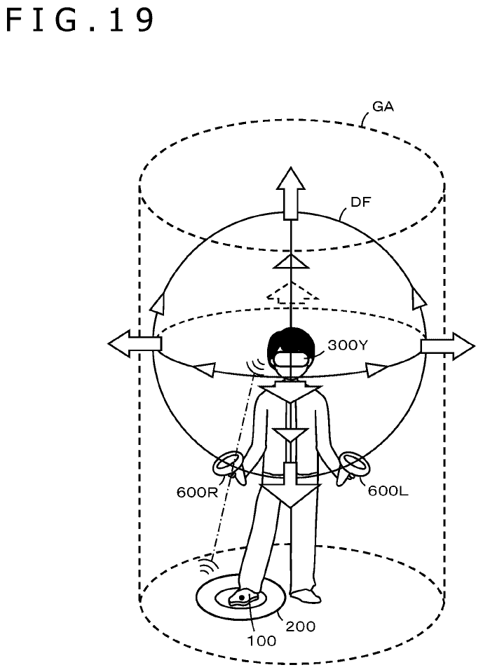

FIG. 19 is a diagram of assistance in explaining the usage of stand-alone VR based on the X-Y-R-axis control using the absolute coordinate system. More specifically, FIG. 19 is a diagram of assistance in explaining the operation state of the foot input system based on the X-Y-R-axis control using the absolute coordinate system and VR application using an HMD 300Y that supports a stand-alone operation. In an example illustrated in FIG. 19, the foot position detecting device 200 and the HMD 300Y are directly connected to each other wirelessly. The image processing system illustrated in FIG. 19 is also based on the X-Y-R-axis control using the absolute coordinate system, and therefore has similar features in terms of basic configuration to those of the image processing system illustrated in FIG. 18.

However, while the HMD 300Y has a similar external appearance to that of the HMD 300 described above, the HMD 300Y includes a microprocessor having a high processing power, and thus also has functions as the image processing device 500. That is, the HMD 300Y can perform a function of executing software for VR content, a stereoscopic video processing function, and tracking and input processing of the positions and angles of the HMD 300Y and the VR hand controllers 600L and 600R.

Also in FIG. 19, a 360-degree image region GA indicated by a cylindrical body of a dotted line to surround the user corresponds to a range reachable by the hands of the user when the user moves about the foot position detecting device 200. In other words, the 360-degree image region GA corresponds to a range in which there is a possibility of the user physically colliding with an object or a wall during the usage of the image processing system formed using the foot input system according to the present embodiment. In addition, a range indicated by a spherical body DF covering an upper body of the user and a space over the head of the user represents a VR space, which corresponds to a linear movement and a rotational movement on the three axes, that is, the X-axis, the Y-axis, and the Z-axis. That is, the spherical body DF is a 6DoF (Degree of Freedom) VR space that handles not only the “rotation and inclination” of the head and the neck but also forward/rearward, left/right, and upward/downward movements.

Because an external PC is not required to perform the function of the image processing device 500 to execute the software for the VR content, VR application becomes possible in a smallest space, wherein the user's standing position does not get gradually shifted. In the image processing system illustrated in FIG. 19, a hand operation within the VR space is performed by using the VR controllers 600L and 600R, but other configurations are possible. Hand tracking may be used, or a small input terminal as a button input controller may be held in the hands or by a body part other than the hands. In addition, the foot position detecting device 200 may be connected via wire to an outlet or a universal serial bus (USB) port for a purpose of power supply to obtain power for an operation.

### Other Examples of Foot Position Indicating Instrument and Foot Position Detecting Device

The foot position indicating instrument 100 can be improved or refined in terms of miniaturization, the case of fitting to the foot portion of the user, the enhancement of operability on the foot position detecting device, and the like. In addition, the foot position detecting device 200 can be improved or refined in terms of the simplification of configuration, the addition of a new function, and the like. In the following, description will be made of other examples of the foot position indicating instrument 100 and the foot position detecting device 200 in which these improvements and refinements are made. Incidentally, a foot input system including a foot position indicating instrument 100A and a foot position detecting device 200A to be described in the following can also be used as an input system of the image processing device 500 as described with reference to FIG. 2 and the like.

### Another Example of Foot Position Indicating Instrument

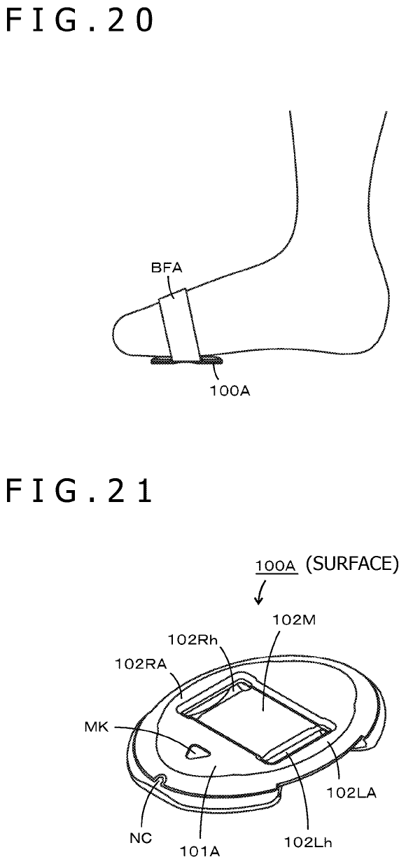

FIG. 20 is a diagram illustrating an example of a state in which the foot position indicating instrument 100A in this example is fitted to the foot portion of the user. The foot position indicating instrument 100A in the present example is more miniaturized than the foot position indicating instrument 100 described with reference to FIGS. 3A to 3C and the like. Therefore, as illustrated in FIG. 20, when the foot position indicating instrument 100A is fitted to a toe side part of the foot portion of the user, for example, the rear side belt is not used, and the foot position indicating instrument 100A can be fitted easily and stably by only one fitting belt BFA corresponding to the front side belt BF in the case of the foot position indicating instrument 100 described above.

In addition, a silicon belt having moderate flexibility and frictional force is used as the fitting belt BFA in the present example. The foot position indicating instrument 100A can be thereby fitted securely without being displaced easily when the foot position indicating instrument 100A is fitted to the foot portion of the user. Furthermore, the foot position indicating instrument 100A can be fitted easily and stably by only one fitting belt BFA even in a case where the foot position indicating instrument 100A is fitted, not to the toe side part but instead, to a plantar arch part or a heel part of the foot portion of the user.

A configuration of the foot position indicating instrument 100A will be described in the following. FIG. 21 is an external view of the foot position indicating instrument 100A, and is a view as viewed from the top surface side (a surface side that comes into contact with the sole of the user) of the foot position indicating instrument 100A. As illustrated in FIG. 21, a main body unit 101A of the foot position indicating instrument 100A has a configuration of a plate-shaped body in a substantially elliptic shape, and is formed to be more elongated than the foot position indicating instrument 100 illustrated in FIGS. 3A to 3C. In addition, as indicated by comparison between FIG. 20 and FIG. 5C, the foot position indicating instrument 100A in the present example is configured such that the length of the foot position indicating instrument 100A in a direction along the longitudinal direction of the foot portion when the foot position indicating instrument 100A is fitted to the foot portion is shorter by approximately 1 to 2 cm, for example, than the foot position indicating instrument 100 described above.

In addition, as illustrated in FIG. 21, an upper surface plate part on the top surface side of the foot position indicating instrument 100A is provided with through holes 102Lh and 102Rh elongated to a degree that the fitting belt BFA passes through the through holes. One portion of the upper surface plate part on the top surface side of the foot position indicating instrument 100A thereby functions as belt holding units 102LA and 102RA. Incidentally, various methods can be used to connect both ends of the fitting belt BFA to each other. The fitting belt BFA is of a silicon material. Thus, for example, both ends thereof may be connected to each other by bonding, fusing, compression bonding, or the like so as not to be separated from each other, and both ends thereof may be connected to each other by using a connecting element.

In addition, a triangular mark MK and a notch NC in the top surface of the main body unit 101A of the foot position indicating instrument 100A indicate that a side provided with the triangular mark MK and the notch NC is oriented to a front side (toe side) in the longitudinal direction of the foot portion. That is, an orientation in which the foot position indicating instrument 100A is fitted to the foot portion of the user is fixed.

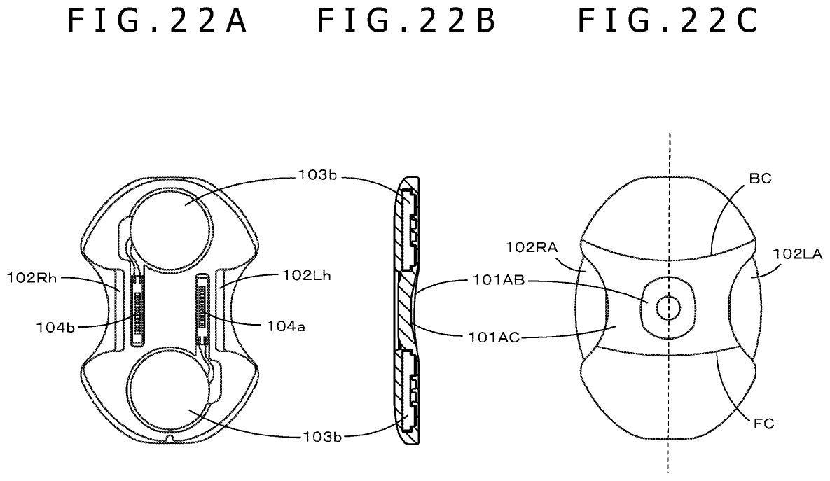

FIGS. 22A to 22C are diagrams of assistance in explaining an internal structure of the foot position indicating instrument 100A in the present example and the shape of an undersurface thereof. Specifically, FIG. 22A is a view of the internal structure of the foot position indicating instrument 100A, FIG. 22B is a sectional view of the foot position indicating instrument 100A, and FIG. 22C is a view illustrating the undersurface side of the foot position indicating instrument 100A. Incidentally, the sectional view illustrated in FIG. 22B is a sectional view in a case where sectioning is performed in a part indicated by a dotted line in the view illustrating the undersurface side of the foot position indicating instrument 100A in FIG. 22C.

As illustrated in FIG. 22A, the internal structure appears when the upper surface plate part of the main body unit 101A of the foot position indicating instrument 100A illustrated in FIG. 21 is removed. As illustrated in FIG. 22A, a casing that is of a substantially elliptic shape but has a shape in which the outer edge of a central part in a longitudinal direction is recessed inward appears on the lower side of the upper surface plate of the main body unit 101A. The casing internally includes a resonance circuit formed of coil 103a formed flat and a resonance circuit board 104a, and a resonance circuit formed of a coil 103b formed flat and a resonance circuit board 104b.

The resonance circuit boards 104a and 104b are formed with circuit parts such as capacitors mounted thereon. In addition, as illustrated in FIG. 22A, in the case of the foot position indicating instrument 100A, through holes 102Lh and 102Rh for fitting the fitting belt BFA to the foot position indicating instrument 100A are provided along the resonance circuit boards 104a and 104b. Incidentally, though not illustrated in FIG. 22A, a midpoint as a middle position of a straight line connecting the center of the coil 103a and the center of the coil 103b to each other is a position detected in the foot position detecting device 200A to be described later.

In addition, the undersurface of the foot position indicating instrument 100A in the present example is provided with an arcuate groove portion 101AC, and a central part of the arcuate groove portion 101AC is provided with a hemisphere recess (recessed portion) 101AB. The arcuate groove portion 101AC is a part to be fitted with a doughnut-shaped convex portion of the foot position detecting device 200A to be described later, and the recess 101AB is a part to be fitted with a protrusion Cp formed in a central part of a position detecting unit 220A of the foot position detecting device 200A to be described later. That is, the undersurface of the foot position indicating instrument 100A is provided with a recessed portion in which the hemisphere recess 101AB and the arcuate groove portion 101AC are superposed on each other to form a recessed shape.

Thus, the foot position indicating instrument 100A in the present example is miniaturized, is allowed to be easily fitted to the foot portion of the user, and further achieves an improvement in operability on the foot position detecting device 200A due to relation to the shape of the position detecting unit of the foot position detecting device 200A to be described later.

### Another Example of Foot Position Detecting Device

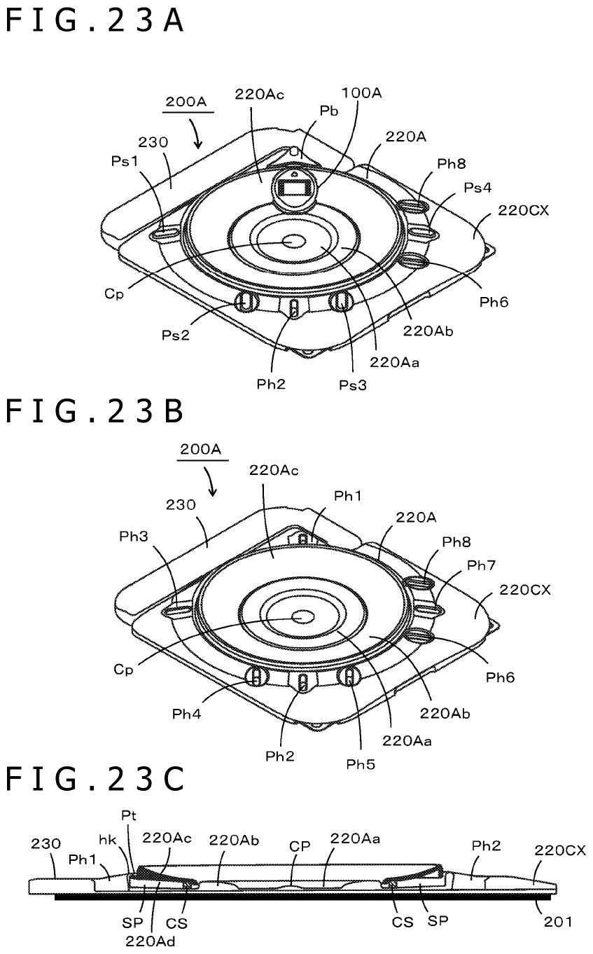

FIGS. 23A to 23C are diagrams of assistance in explaining an external configuration of the foot position detecting device 200A in another example. Of FIGS. 23A to 23C, FIGS. 23A and 23B are perspective views of the foot position detecting device 200A, and FIG. 23C is a sectional view of the foot position detecting device 200. Incidentally, the perspective view of FIG. 23A illustrates a state in which the foot position indicating instrument 100A described above is mounted and a large positioner Pb and small positioners Ps1, Ps2, Ps3, and Ps4 to be described later are fitted. The perspective view of FIG. 23B illustrates a state in which the foot position indicating instrument 100A described above is not mounted, and the large positioner Pb and the small positioners Ps1, Ps2, Ps3, and Ps4 to be described later are not fitted. In FIGS. 23A to 23C, parts formed similarly to those of the foot position detecting device 200 illustrated in FIGS. 6A and 6B are identified by the same reference signs.

That is, the foot position detecting device 200A in the present example also has the internal configuration of the configuration described with reference to FIG. 7, and includes a circuit mounted unit 230 mounted with a position detecting circuit, as illustrated in FIGS. 23A and 23B. In addition, as illustrated in solid black in FIG. 23C, a position detecting sensor 201 connected to the position detecting circuit 202 is provided. The foot position detecting device 200A in the present example is different from the foot position detecting device 200 described with reference to FIGS. 6A and 6B in terms of the configuration of the position detecting unit cover 220CV part.

As illustrated in FIGS. 23A and 23B, the external appearance of the foot position detecting device 200A includes a quadrangular position detecting unit cover 220CX having a large circular position detecting unit 220A formed therein and the circuit mounted unit 230 formed in an L-shape in an upper left part. This is similar to that in the case of the foot position detecting device 200 illustrated in FIGS. 6A and 6B. In addition, as indicated by a plurality of concentric circles, the position detecting unit 220A also has an overall shape like a dish which is depressed (recessed) stepwise from the outside to the inside. In the present embodiment, the position detecting unit 220A has a three-stage structure of a highest outer portion, a lowest inner portion, and an intermediate portion located therebetween.

Specifically, a lowest circular part located in the center of the position detecting unit 220A is a central circular portion (inner portion) 220Aa provided with a protrusion Cp in a central part thereof. On the periphery of the central circular portion 220Aa is a doughnut-shaped convex portion (intermediate portion) 220Ab at a position somewhat higher than the central circular portion 220Aa, the doughnut-shaped convex portion having a predetermined width and slightly bulging upward. On the periphery of the doughnut-shaped convex portion 220b is a ring-shaped inclined portion (outer portion) 220Ac inclined so as to rise from the inside to the outside.

On the thus formed position detecting unit 220A, as illustrated in FIG. 23A, the foot position indicating instrument 100A fitted to the foot portion of the user is positioned and is used so as to be moveable. As illustrated in FIG. 23A, the large positioner Pb is provided on an upper side, the small positioners Ps1 and Ps4 are provided on a left side and a right side, and the small positioners Ps2 and Ps3 are provided on a lower side so as to be laid on the outer edge of the position detecting unit 220A formed in a circular shape.

These positioners correspond to the direction detection protruding portions 221, 222, 223, and 224 of the foot position detecting device 200 described with reference to FIGS. 6A and 6B. In the foot position detecting device 200A illustrated in FIG. 23A, a straight line connecting the central axis of the large positioner Pb and a midpoint between the small positioner Ps2 and the small positioner Ps3 is the Y-axis, and a straight line connecting the central axis of the small positioner Ps1 and the central axis of the small positioner Ps4 is the X-axis. Also in the foot position detecting device 200A, the Y-axis and the X-axis are orthogonal to each other at the center of the position detecting unit 220A. Incidentally, the above description of the X-axis and the Y-axis represents an example of a determining method that is easily understandable by the user but, in actuality, the X-axis and the Y-axis are determined in advance on the position detecting sensor 201 of the foot position detecting device 200A. The X-axis and the Y-axis are set to be easily understandable by the user, as described above.

As illustrated in FIG. 23B, an outer edge portion of the position detecting unit 220A formed in a circular shape in the position detecting unit cover 220CX of the foot position detecting device 200A in the present example is provided with positioner attachment holes Ph1, Ph2, Ph3, Ph4, Ph5, Ph6, Ph7, and Ph8. Thus, the user attaches the large positioner and the small positioners at necessary positions among the positioner attachment holes Ph1, Ph2, Ph3, Ph4, Ph5, Ph6, Ph7, and Ph8 in a manner convenient for the user. The user can thus recognize the Y-axis and the X-axis on the position detecting sensor 201. For example, large positioners can be attached to the positioner attachment holes Ph1, Ph2, Ph3, and Ph7, or conversely, small positioners can be attached to the positioner attachment holes Ph1, Ph2, Ph3, and Ph7.

Also in the foot position detecting device 200A in the present example, the position detecting unit cover 220CX is detachable from basic constituent parts of the foot position detecting device 200A including the position detecting sensor 201 and the circuit mounted unit 230 mounted with the position detecting circuit 202. Further, in the case of the foot position detecting device 200A in the present example, the ring-shaped inclined portion 220Ac is configured to be detachable from the position detecting unit cover 220CX.

FIG. 23C is a sectional view of the foot position detecting device 200A sectioned by a straight line (Y-axis) connecting the positioner attachment hole Ph1 and the positioner attachment hole Ph2 in FIG. 23B to each other. As illustrated in FIG. 23C, the position detecting unit cover 220CX in the present example is used in a state of being mounted on the position detecting sensor 201. The position detecting unit cover 220CX has the position detecting unit 220A on the inside of the positioner attachment hole Ph1 and the positioner attachment hole Ph2. A central portion of the position detecting unit cover 220CX in the present example is the central circular portion 220Aa. The doughnut-shaped convex portion 220Ab is formed on the outside of the central circular portion 220Aa. A ring-shaped recessed portion 220Ad is formed in a ring shape on the outside of the doughnut-shaped convex portion 220Ab.

Thus, the circular position detecting unit 220A of the position detecting unit cover 220CX has the central circular portion 220Aa, the doughnut-shaped convex portion 220Ab, and the ring-shaped recessed portion 220Ad concentrically formed in this order from a central portion. Moreover, a configuration is adopted in which the ring-shaped inclined portion 220Ac inclined so as to rise toward the outside is mounted over the outermost ring-shaped recessed portion 220Ad of the position detecting unit 220A. An inner edge portion of the ring-shaped inclined portion 220Ac is provided with a ring-shaped cushion CS along the outer edge of the doughnut-shaped convex portion 220Ab. Incidentally, a configuration may be adopted such that a plurality of springs are arranged along the outer edge of the doughnut-shaped convex portion 220Ab in place of the ring-shaped cushion CS. Consequently, a ring-shaped space SP is formed between the ring-shaped inclined portion 220Ac and the position detecting sensor 201.

The foot position detecting device 200A in the present example formed so as to be mounted with the position detecting unit cover 220CX and the ring-shaped inclined portion 220Ac having such a configuration enables an operation that cannot be realized with the foot position detecting device 200 described with reference to FIGS. 6A and 6B. Specifically, it is possible to perform a position indication so as to move the foot position indicating instrument 100A fitted to the foot portion of the user on the position detecting unit 220A, and perform, on the ring-shaped inclined portion 220Ac, an operation of depressing the ring-shaped inclined portion 220Ac.

Thus, on the ring-shaped inclined portion 220Ac, it is possible to detect the height of the foot position indicating instrument 100A above the position detecting sensor 201, that is, the height of the coils 103a and 103b of the foot position indicating instrument 100A from the position detecting sensor 201. It is thus possible to perform an indication input, for example, to change the movement speed of an avatar or the like in a computer game being executed, for example, according to a degree of depressing the foot position indicating instrument 100A on the ring-shaped inclined portion 220Ac as in a case of an accelerator of an automobile, for example. That is, it is possible to perform an indication input (indication operation) according to a degree of depression, which cannot be performed with the foot input system including the foot position indicating instrument 100 and the foot position detecting device 200 described above.

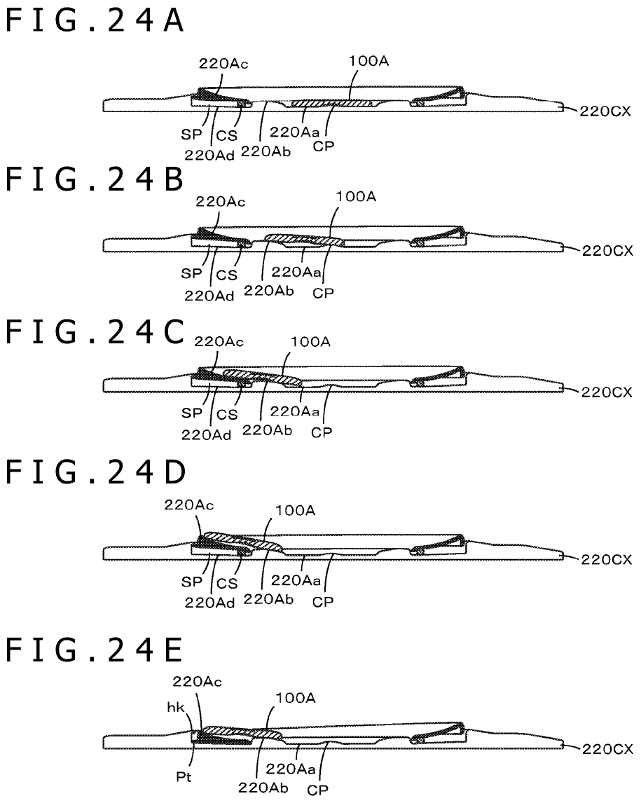

FIGS. 24A to 24E are diagrams of assistance in explaining the operation of the foot position indicating instrument 100A on the foot position detecting device 200A in the present example, and illustrates a section of the position detecting unit cover 220CX of the foot position detecting device 200A and a section of the main body unit 101A of the foot position indicating instrument 100A. Incidentally, in order to clarify a distinction therebetween, the section of the position detecting unit cover 220CX of the foot position detecting device 200A is illustrated in an outlined state, the ring-shaped inclined portion 220Ac as a movable portion is illustrated in solid black, and the section of the main body unit 101A of the foot position indicating instrument 100A is illustrated in a hatched state.

The length in the longitudinal direction of the main body unit 101 of the foot position indicating instrument 100A in the present example is set to be equal to or slightly shorter than the diameter of the central circular portion 220Aa of the foot position detecting device 200A in the present example. In addition, an internal surface shape of the recess 101AB in a hemisphere shape in the foot position indicating instrument 100A and an external surface shape of the protrusion Cp of the central circular portion 220Aa in the foot position detecting device 200A are made to coincide with each other.

Thus, as illustrated in FIG. 24A, suppose that the foot position indicating instrument 100A is located on the central circular portion 220Aa of the position detecting unit 220A of the foot position detecting device 200A. In this case, the recess 101AB of the undersurface of the foot position indicating instrument 100A and the protrusion Cp of the central circular portion 220Aa of the position detecting unit 220A are fitted to and catch (engage) each other. The position of the foot position indicating instrument 100A on the position detecting unit 220A of the foot position detecting device 200A is thereby regulated, so that the user can clearly grasp the position of the user in the real space. Furthermore, a horizontal slipping of the foot position indicating instrument 100A on the position detecting unit 220A is prevented, and the foot position indicating instrument 100A can be rotated smoothly over the entire circumference of 360 degrees. That is, the user fitted with the foot position indicating instrument 100A can smoothly and stably rotate about the foot fitted with the foot position indicating instrument 100A.

In addition, in the position detecting unit 220A of the foot position detecting device 200A, the doughnut-shaped convex portion 220Ab that has a predetermined width and bulges upward is present on the periphery of the central circular portion 220Aa and at a position somewhat higher than the central circular portion 220Aa. Therefore, as illustrated in FIG. 24B, when the foot position indicating instrument 100A is moved outward from the central circular portion 220Aa of the position detecting unit 220A, the doughnut-shaped convex portion 220Ab blocks a sudden movement of the foot position indicating instrument 100A, and allows the foot position indicating instrument 100A to be moved slowly.

Conversely, when the foot position indicating instrument 100A is moved from the doughnut-shaped convex portion 220Ab side to the central circular portion 220Aa side during the state illustrated in FIG. 24B, the foot position indicating instrument 100 can be moved to slide down to the central convex portion 220a. A structure is therefore provided which facilitates a quick return of the foot position indicating instrument 100A located on the doughnut-shaped convex portion 220Ab to the central circular portion 220Aa at the center. This structure allows the foot position indicating instrument 100 to return to the central circular portion 220Aa quickly in any direction over 360 degrees about the central circular portion 220Aa when the foot position indicating instrument 100 is moved from the doughnut-shaped convex portion 220Ab side to the central circular portion 220Aa side.

Thus, the doughnut-shaped convex portion 220Ab located in an intermediate portion of the position detecting unit 220A allows a slow movement from the central circular portion 220Aa to the outside and a quick return from the doughnut-shaped convex portion 220b side to the central circular portion 220Aa. This structure realizes functions equivalent to those of the foot input system including the foot position indicating instrument 100 and the foot position detecting device 200 described above.

Further, in the case of the foot input system including the foot position indicating instrument 100A and the foot position detecting device 200A, the arcuate groove portion 101AC in the undersurface of the foot position indicating instrument 100A is formed in an arcuate shape such that the doughnut-shaped convex portion 220Ab fits exactly into the arcuate groove portion 101AC. That is, a front side outer edge FC of the arcuate groove portion 101AC in the undersurface of the foot position indicating instrument 100A is along an outer depth portion of the doughnut-shaped convex portion 220Ab, and a rear side outer edge BC of the arcuate groove portion 101AC in the undersurface of the foot position indicating instrument 100A is along an inner depth portion of the doughnut-shaped convex portion 220Ab.

Therefore, in a state in which the doughnut-shaped convex portion 220Ab has fitted into the arcuate groove portion 101AC in the undersurface of the foot position indicating instrument 100A, the position of the foot position indicating instrument 100A is maintained stably. Even in a case where the foot position indicating instrument 100A is moved outward from this state, and even in a case where the foot position indicating instrument 100A is moved inward from this state, the foot position indicating instrument 100A is prevented from making a sudden movement such as is not intended by the user, and can be moved stably as intended by the user.

When the foot position indicating instrument 100A goes over the doughnut-shaped convex portion 220Ab from the state illustrated in FIG. 24B, an end of the foot position indicating instrument 100A rubs against the inclined surface of the ring-shaped inclined portion 220Ac, and thus the inclined surface plays a role of a brake, as illustrated in FIG. 24C. That is, a movement of the foot position indicating instrument 100A such that the foot position indicating instrument 100A is shifted to the device side is suppressed, and a braking effect is produced on the foot position indicating instrument 100A, thus facilitating an adjustment for positioning the foot position indicating instrument 100A on the position detecting unit 220A.

When the foot position indicating instrument 100A is further moved outward as illustrated in FIG. 24D from the state illustrated in FIG. 24C, an operation of depressing the ring-shaped inclined portion 220Ac becomes possible. The ring-shaped inclined portion 220Ac is supported by the ring-shaped cushion CS. Therefore, when the user applies no load to the foot portion fitted with the foot position indicating instrument 100A, the ring-shaped inclined portion 220Ac is not depressed, and a position indicated by the foot position indicating instrument 100A can be detected. However, even when the outer edge of the foot position indicating instrument 100A is located in the vicinity of an edge portion of the ring-shaped inclined portion 220Ac as illustrated in FIG. 24D, a maximum value of a detection value (detection output) of the indicated position is not obtained in the foot position detecting device 200A.

As illustrated in FIG. 25E, suppose that the user applies a load to the foot portion fitted with the foot position indicating instrument 100A, and depresses the ring-shaped inclined portion 220Ac via the foot position indicating instrument 100A. The ring-shaped recessed portion 220Ad is formed on the lower side of the ring-shaped inclined portion 220Ac, and the ring-shaped space SP is formed between the ring-shaped inclined portion 220Ac and the ring-shaped recessed portion 220Ad.

In this case, a distance between the foot position indicating instrument 100A and the position detecting sensor 201 is decreased, the space SP is narrowed according to the degree of the pressing, and consequently the detection value (detection output) of the indicated position in the foot position detecting device 200A is increased. As illustrated in FIG. 24E, when the ring-shaped inclined portion 220Ac is depressed to a maximum, the detection output of the foot position detecting device 200A becomes a maximum value. Hence, the level of the detection value of the indicated position changes according to the degree of depressing the ring-shaped inclined portion 220Ac of the position detecting unit 220A of the foot position detecting device 200A through the foot position indicating instrument 100A, and various types of control is enabled by using this change.

For example, as also described above, an output value corresponding to the degree of depressing the ring-shaped inclined portion 220Ac can be used for various types of control such as control of the movement speed of the avatar or the like in game software being executed, the descending and ascending of the avatar or the like, adjustment of a degree of inflation of a balloon object, and the like. Thus, the ring-shaped inclined portion 220Ac enables control according to the detection output corresponding to the distance between the foot position indicating instrument 100A and the position detecting sensor, which distance corresponds to the degree of the depression. That is, the ring-shaped inclined portion 220Ac can be used as a so-called pedal operating unit such as an accelerator pedal of an automobile, for example.

Incidentally, the ring-shaped inclined portion 220Ac is separate from the position detecting unit cover 220CX. Therefore, as illustrated in FIG. 24E, a protrusion pt provided to the outer edge of the ring-shaped inclined portion 220Ac and a protrusion hk protruding inward on an outermost circumferential part of the position detecting unit 220A of the position detecting unit cover 220CX are engaged with each other. The ring-shaped inclined portion 220Ac is thereby prevented from being detached easily.

Thus, the foot position detecting device 200A in the present example does not include the outer wall-shaped protruding portion 202c but includes the ring-shaped inclined portion 220Ac that can be depressed. The foot position detecting device 200A thereby also enables an input by an operation of depressing the ring-shaped inclined portion 220Ac. The foot position detecting device 200A and the foot position indicating instrument 100A can realize a foot input system provided with a new input function. In addition, the foot input system constituted by the foot position indicating instrument 100A and the foot position detecting device 200A can realize input functions equivalent to those of the foot input system including the foot position indicating instrument 100 and the foot position detecting device 200 described above except that the operation of depressing the ring-shaped inclined portion 220Ac can be performed.

Incidentally, also the position detecting unit cover CX in the present example is detachable from basic constituent parts (basic casing parts) of the foot position detecting device 200 including the position detecting sensor 201 and the circuit mounted unit 230 mounted with the position detecting circuit 202. That is, the position detecting unit cover 220CX including the position detecting unit 220A has a configuration of an attachment as an accessory part in the foot position detecting device 200A. Further, in the case of the present example, the ring-shaped inclined portion 220Ac is detachable from the position detecting unit cover 220CX. Needless to say, the ring-shaped inclined portion 220Ac does not have to be detachable from the position detecting unit cover 220CX. That is, it suffices for the ring-shaped inclined portion 220Ac part to be formed in such a manner as to be depress-able.

### Effects of Embodiments

As is understood from the description of the foregoing embodiments, the foot input system used in the image processing system can change values on three axes of the X-axis, the Y-axis, and the R-axis, two axes of the X-axis and the Y-axis, and one axis of the Y-axis in a manner similar to the analog axes of a joystick, for example. Thus, the foot input device according to the foregoing embodiments can be used as a game controller. The foot input system according to the foregoing embodiments enables a fine indication input to be performed according to a fine movement of the foot position indicating instrument 100, rather than an indication input for on/off switching.

In addition, the user can naturally move the body in a moving direction at a time of an operation due to the usage of the foot input system. It is thereby possible to suppress VR sickness. Further, it is possible to not only use change in the value on each axis as a linear output but also convert the change into a nonlinear output obtained by performing quadratic function complementation or cubic function complementation, for example, or provide the change with hysteresis. This may further suppress the occurrence of VR sickness.

In addition, the foot position indicating instrument 100 is usable only on the position detecting unit 220 of the foot position detecting device 200, and both of their shapes can regulate (limit) the position of the user. That is, the VR technology can be used in a reduced space with one fixed point as a center thereof. Thus, the user can properly understand the user's position and avoid getting entangled with a cable or running into a wall, for example.

In addition, the usage of the foot input system allows the hands and the foot to move separately to serve different roles, so that an operation is not complex. That is, a hand operation in the VR space by the real hands and a movement operation in the VR space by the real foot are separate from each other, and the position movement within the VR space can be performed (by the foot) without the VR controller held in the hands, to simplify the operation. Simply stated, the foot input system is used for movement operations of the avatar and the viewpoint, and operations using a hand controller or hand tracking, for example, can be performed as other input operations.

Because movements performed by the hands and the foot for different purposes can be performed separately, and usable postures are not limited, and operations can be performed by minimum movements with a low delay in a high resolution such as putting the foot forward, rearward, left, or right and turning the foot, a high sense of immersion can be achieved. That is, the sense of immersion is not impaired when using the VR technology.

In addition, in the case of the foot input system according to the foregoing embodiments, there is no need for a large device body, and the foot position indicating instrument 100 and the foot position detecting device 200 can realize a foot input system (a multiple axis control controller) in a casing of a compact size that can be fitted in a bookshelf, for example. In addition, the foot input system including the foot position indicating instrument 100 and the foot position detecting device 200 does not require repeated stepping actions, does not limit usable postures, does not cause an input delay, and enables a fine operating input. Hence, various challenges facing the existing movement operation devices using a foot portion (leg portion) can be eliminated.

### Modifications

It is to be noted that, while the position detecting unit 220 of the foot position detecting device 200 has a three-stage structure of the outer wall-shaped protruding portion (outer portion) 220c, the doughnut-shaped convex portion (intermediate portion) 220b, and the central convex portion (inner portion) 220a in the foregoing embodiments, the position detecting unit 220 is not so limited. A multiple stage configuration of four stages or more can obviously be adopted.

In addition, in the foregoing embodiments, the position detecting unit 220 of the foot position detecting device 200 has been described as one having a circular dish shape. However, the position detecting unit 220 is not so limited. For example, it can be formed in a polygonal shape such as a quadrangular shape, a pentagonal shape, or a hexagonal shape. In addition, in the foregoing embodiments, the foot position indicating instrument 100 has been described as one having a substantially circular shape. However, it is not so limited. The foot position indicating instrument 100 can also be formed in a polygonal shape such as, for example, a quadrangular shape, a pentagonal shape, or a hexagonal shape.

In addition, in the foregoing embodiments, description has been made supposing that the HMD 300, 300X, or 300Y is used as a display device (output device). However, the embodiments are not so limited. As the display device, a display device of an installation type such as a television receiver can be used, or a display device of a projection type that projects video onto a screen can be used. Various other display devices can be used as the display device.

In the foregoing embodiments, the foot position indicating instrument 100 includes resonance circuits, as described with reference to FIGS. 3A to 3C. In addition, as described with reference to FIG. 7, the foot position detecting device 200 includes a position detecting sensor formed by arranging a plurality of loop coils in the X-axis direction and the Y-axis direction. That is, the foot input system including the foot position indicating instrument 100 and the foot position detecting device 200 is of an electromagnetic induction type that allows a position indication or the like to be performed through a magnetic field. Therefore, the foot position indicating instrument 100 does not need to be mounted with a battery and can be reduced in size and reduced in weight. However, the foot position indicating instrument 100 is not so limited.

For example, the foot position indicating instrument is mounted with a battery and a position indication signal transmitting unit, and the foot position detecting device is mounted with a position detecting sensor formed by arranging a plurality of line electrodes in the X-axis direction and the Y-axis direction. It is thereby possible to form a foot input system of a so-called active capacitive type. That is, the present disclosure relates to position indications, and the present disclosure is applicable to foot input systems including foot position indicating instruments and foot position detecting devices of various types.

The foot position detecting device 200A of one example of the foregoing embodiments includes the ring-shaped inclined portion 220Ac as a movable portion that can be depressed. However, the disclosure is not so limited. A foot position detecting device can be formed which has a configuration such that the central circular portion 220Aa, the doughnut-shaped convex portion 220Ab, and the ring-shaped inclined portion 220Ac are integrally formed as a position detecting unit cover, and thus the ring-shaped inclined portion 220Ac is not formed as a movable portion. The foot position detecting device of this configuration has functions equivalent to those of the foot position detecting device 200 described above. Furthermore, the configuration can be simplified by including the immovable ring-shaped inclined portion 220Ac rather than the outer wall-shaped protruding portion 220c, to suppress an unintentional outward movement of the foot position indicating instrument 100 or 100A.

Of indications of movement directions within the VR space, indications of movement directions by a rotational movement are more common than indications of movement by a horizontal movement in a left/right direction. In terms of the case of movement of a foot of a human, a horizontal movement that moves the foot left or right can be performed more easily than a rotational movement that turns the foot left or right. This is felt particularly when the foot position indicating instrument 100 or 100A is fixed to the toe side. Accordingly, operability can be improved by interchanging, with respect to the rotational movement and the left/right movement of the foot, the output values of the rotational movement indication using the foot and the output values of the left/right movement indication using the foot.

That is, an output corresponding to an operation of turning the foot to the right from the foot position detecting device 200 or 200A is recognized and processed as indication information for a horizontal movement in the right direction in the image processing device 500. Conversely, an output corresponding to an operation of moving the foot portion horizontally in the right direction from the foot position detecting device 200 or 200A is recognized and processed as indication information for a rotational movement in the right direction in the image processing device 500. It is thereby possible to improve operability in the case of using the foot input system including the foot position indicating instrument 100 and the foot position detecting device 200 or the foot input system including the foot position indicating instrument 100A and the foot position detecting device 200A.

1. A foot input system, comprising:
a foot position indicating instrument configured to be located on a sole of a user and including one or more position indication signal transmitting units configured to transmit a position indication signal; and
a foot position detecting device including:
a position detecting unit that forms an operation surface on which the foot position indicating instrument moves and that receives a position indicated by the foot position indicating instrument;
a position detecting sensor provided on a lower side of the position detecting unit to detect the position indicated by the foot position indicating instrument on a surface of the position detecting unit; and
a detecting circuit configured to be supplied with a detection output from the position detecting sensor and detect and output the position indicated by the foot position indicating instrument on the position detecting unit;
wherein, the position detecting unit has a concentric uneven structure.

2. The foot input system according to claim 1, wherein
the position detecting unit has a three-stage structure including, from an outside to an inside, an outer portion, an intermediate portion, and an inner portion.

3. The foot input system according to claim 2, wherein
the outer portion constitutes an outer wall-shaped protruding portion forming an outer wall along an outer edge, the intermediate portion constitutes a doughnut-shaped convex portion bulging upward, and the inner portion constitutes a central convex portion that bulges upward in a spherical shape, and
a bottom surface of the foot position indicating instrument forms a spherical recessed portion to be fitted with the central convex portion.

4. The foot input system according to claim 2, wherein
the outer portion constitutes a ring-shaped inclined portion inclined so as to rise from the inside to the outside, the intermediate portion constitutes a doughnut-shaped convex portion bulging upward, and the inner portion constitutes a central circular portion formed in a circular shape and having a hemispherical protrusion provided at a center, and
a bottom surface of the foot position indicating instrument includes an arcuate groove portion to be fitted with the doughnut-shaped convex portion and a spherical recessed portion to be fitted with the hemispherical protrusion of the central circular portion.

5. The foot input system according to claim 2, wherein
the outer portion constitutes a ring-shaped inclined portion inclined so as to rise from the inside to the outside, the intermediate portion constitutes a doughnut-shaped convex portion bulging upward, and the inner portion constitutes a central circular portion formed in a circular shape and having a hemispherical protrusion provided at a center,
the ring-shaped inclined portion is separate from the doughnut-shaped convex portion and is capable of being depressed downward, and
a bottom surface of the foot position indicating instrument includes an arcuate groove portion to be fitted with the doughnut-shaped convex portion and a spherical recessed portion to be fitted with the hemispherical protrusion of the central circular portion.

6. The foot input system according to claim 1, wherein
the position detecting unit is provided with four direction detection protruding portions at intervals of 90 degrees.

7. The foot input system according to claim 1, wherein
the position detecting unit is detachably provided on the position detecting sensor.

8. The foot input system according to claim 1, wherein
the foot position indicating instrument includes two position indication signal transmitting units having centers located on a straight line in a longitudinal direction of the sole of the user, and
the detecting circuit of the foot position detecting device detects a midpoint position between the two position indication signal transmitting units as the position indicated by the foot position indicating instrument.

9. The foot input system according to claim 1, wherein
the detecting circuit of the foot position detecting device detects the position indicated by the foot position indicating instrument and a rotational angle of the foot position indicating instrument with respect to a defined reference axis in an absolute coordinate system defined on the position detecting unit.

10. The foot input system according to claim 1, wherein
the detecting circuit of the foot position detecting device detects a change in a Y-axis direction and a change in an X-axis direction in a relative coordinate system defined according to an orientation of the foot position indicating instrument on the position detecting unit.

11. The foot input system according to claim 1, wherein
the detecting circuit of the foot position detecting device detects only a change in a Y-axis direction in a relative coordinate system defined according to an orientation of the foot position indicating instrument on the position detecting unit.

12. The foot input system according to claim 1, wherein
the detecting circuit of the foot position detecting device is operable in:
a first detection mode of detecting the position indicated by the foot position indicating instrument and a rotational angle of the foot position indicating instrument with respect to a defined reference axis in an absolute coordinate system defined on the position detecting unit,
a second detection mode of detecting a change in a Y-axis direction and a change in an X-axis direction in a relative coordinate system defined according to an orientation of the foot position indicating instrument on the position detecting unit, and
a third detection mode of detecting only a change in the Y-axis direction in the relative coordinate system defined according to the orientation of the foot position indicating instrument on the position detecting unit, and
the foot position detecting device is configured to receive an input to select which of the first detection mode, the second detection mode, or the third detection mode to use.

13. A foot position detecting device, for use in conjunction with a foot position indicating instrument configured to be located on a sole of a user, the foot position detecting device comprising:
a position detecting unit that forms an operation surface on which the foot position indicating instrument moves and that receives a position indicated by the foot position indicating instrument;
a position detecting sensor provided on a lower side of the position detecting unit to detect the position indicated by the foot position indicating instrument on a surface of the position detecting unit; and
a detecting circuit configured to be supplied with a detection output from the position detecting sensor and detect and output the position indicated by the foot position indicating instrument on the position detecting unit,
wherein, the position detecting unit has a concentric uneven structure.

14. The foot position detecting device according to claim 13, wherein
the position detecting unit has a three-stage structure including, from an outside to an inside, of an outer portion, an intermediate portion, and an inner portion.

15. The foot position detecting device according to claim 14, wherein
the outer portion constitutes an outer wall-shaped protruding portion forming an outer wall along an outer edge, the intermediate portion constitutes a doughnut-shaped convex portion bulging upward, and the inner portion constitutes a central convex portion that bulges upward in a spherical shape.

16. The foot position detecting device according to claim 14, wherein
the outer portion constitutes a ring-shaped inclined portion inclined so as to rise from the inside to the outside, the intermediate portion constitutes a doughnut-shaped convex portion bulging upward, and the inner portion constitutes a central circular portion formed in a circular shape and having a hemispherical protrusion provided at a center.

17. The foot position detecting device according to claim 14, wherein
the outer portion constitutes a ring-shaped inclined portion inclined so as to rise from the inside to the outside, the intermediate portion constitutes a doughnut-shaped convex portion bulging upward, and the inner portion constitutes a central circular portion formed in a circular shape and having a hemispherical protrusion provided at a center, and
the ring-shaped inclined portion is separate from the doughnut-shaped convex portion and is capable of being depressed downward.

18. A foot position indicating instrument, for use in conjunction with a foot position detecting device and configured to be located on a sole of a user, the foot position indicating instrument comprising:
a main body unit having a bottom surface including one or more recessed portions to be respectively fitted with corresponding protruding portions of the foot position detecting device; and
one or more position indication signal transmitting units configured to transmit a position indication signal.

19. The foot position indicating instrument according to claim 18, comprising:
one or more belt holding units.

20. The foot position indicating instrument according to claim 18, wherein the main body unit is formed of a polyacetal (POM) resin.
